(19) United States
(12) Patent Application Publication
(10) Pub. No.: US 2023/0181684 A1
(43) Pub. Date: Jun. 15, 2023

(54) NOVEL ANTIBIOTIC COMPOSITIONS AND METHODS OF MAKING OR USING THE SAME

(71) Applicant: DUKE UNIVERSITY, Durham, NC (US)

(72) Inventors: Kelsey T. MORGAN, Durham, NC (US); Dewey G. MCCAFFERTY, Durham, NC (US)

(21) Appl. No.: 17/926,461

(22) PCT Filed: May 19, 2021
(86) PCT No.: PCT/US2021/033157

§ 371 Date: Nov. 18, 2022

Related U.S. Application Data
(60) Provisional application No. 63/026,765, filed on May 19, 2020.

(51) Int. Cl. A61K 38/16; C12N 15/10; C12N 1/20

(52) U.S. Cl. CPC A61K 38/164; C12N 15/1058; C12N 15/1089; C12N 1/20

(57) ABSTRACT

The present disclosure provides methods of identifying source organisms for antibiotic agents and methods of producing novel antibiotic agents. In particular, the disclosure provides methods of identifying novel source organisms for antibiotic agents by sing functionally significant structural motifs to select probes, and mining genome sequences using the selected probes to identify suitable source organisms for production and isolation of novel antibiotic agents.

### CROSS-REFERENCE TO RELATED APPLICATION

This application claims priority to U.S. Provisional Patent Application No. 63/026,765, filed May 19, 2020, which is incorporated herein by reference in its entirety.

### BACKGROUND

For decades antimicrobial chemotherapy has been utilized successfully for the treatment of infectious disease. However, over the past thirty years, the rate of introduction of new-in-class antibiotics has flattened while the rate of clinical cases of infections due to bacteria that are resistant to front-line antibiotics has steadily increased, thus signaling a pressing need for the discovery and development of new antibiotic therapeutics.

Historically, natural products have helped meet this unmet need by providing a rich source of antimicrobial leads, as almost 70% of clinically approved antibiotics are natural products or second-generation natural product derivatives. For example, the glycopeptide antibiotics vancomycin and teicoplanin are first-generation natural products that have efficacy in their native form against infections from Gram-positive pathogens. Unfortunately, many first-generation natural products that possess good antimicrobial activity in vitro fail to make the jump to drug candidates. This failure is due to several possible limitations, including drug stability, poor absorption, toxicity, limited routes of delivery, and/or encounter resistance mechanisms. This creates a paradox in which these liabilities can preclude further investments in second-generation versions. This is a major issue, as second-generation versions may have favorable properties to help overcome initial limitations, as exemplified by second-generation semisynthetic glycopeptides such as telavancin, oritavancin, and dalbavancin that exhibit markedly improved pharmacological properties and reduced toxicity profiles over the parent natural products.

Accordingly, what is needed are methods of identifying novel sources of antibiotic agents, which may be employed to assist in the development of optimized second-generation antibiotics.

### SUMMARY

In some aspects, provided herein are methods for selecting a source organism of an antibiotic agent. In some embodiments, the methods described herein facilitate the identification of novel source organisms of an antibiotic agent. In some embodiments, the method comprises identifying a plurality of functionally significant structural motifs within at least one parent antibiotic agent. A functionally significant structural motif may be a protein that is important for a given function of the parent antibiotic agent. For example, a functionally significant structural motif may be a protein important for antimicrobial activity of the parent antibiotic agent. Alternatively, a functionally significant structural motif may be a region of a protein (e.g. a domain, a subdomain, etc.) that is important for the given function, such as for the antimicrobial activity of the antibiotic agent.

In some embodiments, the least one parent antibiotic agent is a lipodepsipeptide antibiotic agent. For example, the at least one parent antibiotic agent may be a ramoplanin family antibiotic. In some embodiments, the parent antibiotic agent is ramoplanin. In some embodiments, the parent antibiotic agent is enduracidin. In some embodiments, the functionally significant structural motifs are shared in two or more parent antibiotic agents. For example, the functionally significant structural motifs may be shared in ramoplanin and enduracidin.

In some embodiments, the plurality of functionally significant structural motifs comprise at least two of NRPS A, NRPS B, NRPS C, NRPS D, the terminal thioesterase subdomain from NRPS C, FAAL, or ACP. In some embodiments, at least three functionally significant structural motifs are identified. In some embodiments, at least five functionally significant structural motifs are identified. For example, at least two, at least three, at least four, at least five, at least six, or all seven of the above-listed functionally significant structural motifs may be identified. Additionally functionally significant structural motifs may be used in addition to any of the motifs listed above. In some embodiments, the plurality of functionally significant structural motifs comprise each of NRPS A, NRPS B, NRPS C, NRPS D, the terminal thioesterase subdomain from NRPS C, FAAL, and ACP.

In some embodiments, the method further comprises selecting a plurality of probes, wherein each probe comprises a nucleotide sequence encoding an identified functionally significant structural motif or an amino acid sequence of an identified functionally significant structural motif. In some embodiments, one or more probes comprises a nucleotide sequence and one or more probes comprise an amino acid sequence. For example, one or more probes may comprise a nucleotide sequence encoding an identified functionally significant structural motif, and/or one or more probes may comprise an amino acid sequence of an identified functionally significant structural motif.

In some embodiments, the method further comprises identifying homologous proteins having at least 50% sequence identity to at least one probe or to the functionally significant structural motif encoded by at least one probe. In some embodiments, the method further comprises selecting a source organism when the source organism comprises at least three homologous proteins. In some embodiments, the method comprises selecting a source organism when the source organism comprises at least four homologous proteins. In some embodiments, multiple source organisms are identified using the methods described herein. The source organism(s) may represent a viable source for producing an antibiotic agent.

In some embodiments, the method further comprises determining whether the homologous proteins form a biosynthetic gene cluster. In some embodiments, determining whether the homologous proteins form a biosynthetic gene cluster comprises obtaining whole genome sequences for each selected source organism, assembling a sequence similarity network comprising each whole genome sequence, and determining whether a biosynthetic gene cluster is present within the sequence similarity network.

In some embodiments, the method further comprises culturing at least one selected source organism to produce the antibiotic agent, and isolating the antibiotic agent from culture. The antibiotic agent may be purified, and may be subsequently used in a method for treating a bacterial infection in a subject. In some embodiments, the method comprise culturing the selected source organism if the organism is determined to have a biosynthetic gene cluster that facilitates production of lipodepsipeptides.

In some embodiments, culturing the selected source organism results in production of a lipodepsipeptide antibiotic agent. For example, the antibiotic agent produced may be a ramoplanin congener. In some embodiments, the antibiotic agent produced is chersinamycin.

In some aspects, described herein are methods of producing an antibiotic agent. The method comprises selecting a source organism by a method described herein, and subsequently culturing the selected source organism to produce the antibiotic agent. For example, the method may comprise identifying a plurality of functionally significant structural motifs within at least one parent antibiotic agent, developing a plurality of probes, wherein each probe comprises a nucleotide sequence encoding an identified functionally significant structural motif or an amino acid sequence of an identified functionally significant structural motif, identifying homologous proteins having at least 50% sequence identity to at least one probe or to the functionally significant structural motif encoded by at least one probe, selecting a source organism when the source organism comprises at least three homologous proteins, and culturing at least one selected source organism to produce the antibiotic agent.

In some embodiments, the least one parent antibiotic agent is a lipodepsipeptide antibiotic agent. For example, the at least one parent antibiotic agent may be a ramoplanin family antibiotic. In some embodiments, the parent antibiotic agent is ramoplanin. In some embodiments, the parent antibiotic agent is enduracidin. In some embodiments, the functionally significant structural motifs are shared in two or more parent antibiotic agents. For example, the functionally significant structural motifs may be shared in ramoplanin and enduracidin.

In some embodiments, the plurality of functionally significant structural motifs comprise at least two of NRPS A, NRPS B, NRPS C, NRPS D, the terminal thioesterase subdomain from NRPS C, FAAL, or ACP. In some embodiments, at least three functionally significant structural motifs are identified. In some embodiments, at least five functionally significant structural motifs are identified. For example, at least two, at least three, at least four, at least five, at least six, or all seven of the above-listed functionally significant structural motifs may be identified. Additionally functionally significant structural motifs may be used in addition to any of the motifs listed above. In some embodiments, the plurality of functionally significant structural motifs comprise each of NRPS A, NRPS B, NRPS C, NRPS D, the terminal thioesterase subdomain from NRPS C, FAAL, and ACP.

In some embodiments, the method further comprises selecting a plurality of probes, wherein each probe comprises a nucleotide sequence encoding an identified functionally significant structural motif or an amino acid sequence of an identified functionally significant structural motif. In some embodiments, one or more probes comprises a nucleotide sequence and one or more probes comprise an amino acid sequence. For example, one or more probes may comprise a nucleotide sequence encoding an identified functionally significant structural motif, and/or one or more probes may comprise an amino acid sequence of an identified functionally significant structural motif.

In some embodiments, the method further comprises identifying homologous proteins having at least 50% sequence identity to at least one probe or to the functionally significant structural motif encoded by at least one probe. In some embodiments, the method further comprises selecting a source organism when the source organism comprises at least three homologous proteins. In some embodiments, the method comprises selecting a source organism when the source organism comprises at least four homologous proteins. In some embodiments, multiple source organisms are identified using the methods described herein. The source organism(s) may represent a viable source for producing an antibiotic agent.

In some embodiments, the method further comprises determining whether the homologous proteins form a biosynthetic gene cluster. In some embodiments, determining whether the homologous proteins form a biosynthetic gene cluster comprises obtaining whole genome sequences for each selected source organism, assembling a sequence similarity network comprising each whole genome sequence, and determining whether a biosynthetic gene cluster is present within the sequence similarity network.

In some embodiments, the method further comprises culturing at least one selected source organism to produce the antibiotic agent, and isolating the antibiotic agent from culture. The antibiotic agent may be purified, and may be subsequently used in a method for treating a bacterial infection in a subject. In some embodiments, the method comprise culturing the selected source organism if the organism is determined to have a biosynthetic gene cluster that facilitates production of lipodepsipeptides.

In some embodiments, the method further comprises isolating the antibiotic agent from culture. In some embodiments, the method further comprises purifying the isolated antibiotic agent.

In some embodiments, the antibiotic agent produced is a lipodepsipeptide antibiotic agent. In some embodiments, the antibiotic agent produced is a ramoplanin congener. For example, in some embodiments the antibiotic agent produced is chersinamycin.

In some aspects, provided herein are ramoplanin congeners. The ramoplanin congeners may be produced by any suitable method described herein. In some embodiments, provided herein are ramoplanin congeners for use in a method of treating bacterial infection in a subject. In some embodiments, the bacterial infection is an infection associated with one or more Gram-positive bacterium. For example, in some embodiments, the infection is associated with Staphylococcus aureus, Staphylococcus epidermis, Staphylococcus saprophyticus, Staphylococcus haemolyticus, Staphylococcus hominis, Staphylococcus lugdunensis, Streptococcus pneumoniae, Streptococcus pyrogenes, Streptococcus agalactiae, Enterococcus faecium, Enterococcus faecalis, Bacillus anthracis, Bacillus cereus, Clostridium botulinum, Clostridium perfringens, Clostridium difficile, Clostridium tetani, Listeria monocytogenes, or Corynebacterium diptheria. In some embodiments, the ramoplanin congener is chersinamycin.

### BRIEF DESCRIPTION OF THE DRAWINGS

FIG. 1 is a schematic showing the ramoplanin family of antibiotics.

FIG. 2 is a schematic showing one embodiment of a method for the expansion of the ramoplanin family of antibiotics through targeted genome mining. A) Biosynthetic proteins and protein subdomains were selected from the ramoplanin and enduracidin BGCs and used as search queries for a targeted BLASTp search. Initial hits from the BLASTp search were moved forward to identify full gene clusters. B) Bacterial strains identified from SAR-based genome mining were screened for antibiotic production.

FIG. 3 is a sequence similarity network of open reading frames surrounding NRPS proteins in new bacterial strains. The network is assembled for thirteen preliminary strains established through protein Blast analysis (listed in Table 1) with an E value limit of 10⁻⁵ and alignment score of 50. Proteins belonging to strains that were carried forward in further bioinformatic analyses are indicated in teal.

FIG. 4 is a schematic showing condensed sequence similarity network for proteins within the BGCs of ramoplanin, enduracidin, and the five new ramoplanin family BGCs identified in this study. The network is assembled with an E value limit of 10⁻⁵ and alignment score of 50 (solid edges) or 25 (dashed edges).

FIG. 5A is a schematic showing open reading frame comparisons and FIG. 5B is a schematic showing NRPS domain comparisons between ramoplanin family gene clusters. (1) A. ramoplanifer strain ATCC 33076 (ramoplanin), (2) S. fungicidicus strain ATCC 21013 (enduracidin), (3) M. chersina strain DSM 44151 (chersinamycin), (4) A. orientalis strain B-37, (5) A. orientalis strain DSM 40040, (6) A. balhimycina strain FH189, and (6) Streptomyces sp. TLI-053. Amino acids depicted for ramoplanin, enduracidin, and chersinamycin have been confirmed while those for the four remaining strains are based on predictions from conserved adenylation domain specificity sequences. Bolded residues highlight conserved residues relative to ramoplanin. Residues indicated with an “X” could not be predicted. An asterisk denotes a characterized chlorinated residue, though the adenylation domain confers specificity for Hpg.

FIG. 6 shows phylogenetic relationships between NRPS condensation domains. Clusters are colored by C domain subtype: conventional ^{L}CL domains for L-amino acid incorporation, dual C/E domains for D-amino acid incorporation, and starter C domains for N-acyl lipid attachment. Domains in bold correspond to the C domains for characterized peptides ramoplanin, enduracidin, and chersinamycin.

FIG. 7 shows the structure and biosynthetic gene cluster of chersinamycin. A) ORF arrow diagram depicting the defined BGC from chersinamycin based on the generated SSN, and architecture of the four NRPSs within the chersinamycin BGC. Predicted amino acids based on adenylation domain specificity sequences are listed. No residue could be predicted for module 4 of the third NRPS by sequence alone. B) Structure of chersinamycin as supported by bioinformatics and classical structure elucidation efforts. Structural motifs are colored according to the corresponding biosynthetic proteins responsible for their synthesis and incorporation. C) Comparison of biosynthetic enzymes found within the BGCs of chersinamycin, ramoplanin, and enduracidin.

FIG. 8. Confirmation of the chersinamycin gene cluster. A) CRISPR-Cas9 facilitated knockout of five genes within the biosynthetic pathway of chersinamycin. The genes have homology to PLP-dependent aminotransferase (Chers 29), DpgD (Chers 30), DpgC (Chers 31), DpgB (Chers 32), and DpgA (Chers 33). B) Confirmation of the knockout region in APKS7 strain visualized by a 2.2 kb band generated from PCR of gDNA with primers flanking the knockout region. C) Extracted ion chromatograms for the doubly charged ion species of chersinamycin (m/z=1288) in a chersinamycin standard and crude extracts from wild-type M. chersina, APKS7, and APKS7 complemented with 1 mM Dpg.

FIG. 9. Phylogenetic relationship between terminal NRPS C thioesterase domains. Bolded letters indicate confirmed amino acids in enduracidin, ramoplanin, and chersinamycin.

FIG. 10. MS/MS fragmentation of acyclic chersinamycin (b- and y-ion series). The observed ions are shown in blue. An asterisk denotes fragments that were only observed with the loss of sugar units.

FIG. 11A-11B show determination of absolute configuration of amino acids by advanced Marfey's analysis.

FIG. 12. MS/MS spectrum of acyclic chersinamycin showing the diagnostic fragmentation pattern of b- and y-ions. Inlaid figure shows COSY/TOCY (red) and NOESY correlations (blue) for a key region of Dpg13-Chp17, which differs significantly from ramoplanin.

FIG. 13. ¹H NMR (800 MHz, 4:1 H₂O/DMSO-d₆) spectrum of chersinamycin.

FIG. 14. HR-ESI-MS of chersinamycin

FIG. 15. HR-ESI-MS of acyclic chersinamycin.

FIG. 16. ESI-MS spectrum of propionylated-ornithine-chersinamycin.

FIG. 17. MALDI-MS spectrum of hydrogenated ramoplanin (left) and chersinamycin (right). The mass spectrum of hydrogenated ramoplanin (bottom) exhibits a clear 4 Da shift from starting material (top). The mass spectra for chersinamycin starting material (top) and hydrogenated product (bottom) are identical suggesting a saturated N-acyl lipid.

FIG. 18. ESI-MS/MS spectrum of chersinamycin.

FIG. 19. ESI-MS/MS spectrum of acyclic chersinamycin.

FIG. 20. ¹H-¹H COSY (800 MHz, 4:1 H₂O/DMSO-d₆) spectrum of chersinamycin.

FIG. 21. ¹H-¹H TOCSY (800 MHz, 4:1 H₂O/DMSO-d₆) spectrum of chersinamycin.

FIG. 22. ¹H-¹H NOESY (800 MHz, 4:1 H₂O/DMSO-d₆) spectrum of chersinamycin.

FIG. 23. ¹H-¹H NOESY (800 MHz, D₂O/DMSO-d₆) spectrum of chersinamycin.

FIG. 24. Depiction of defining NMR correlations observed in chersinamycin. COSY/TOCSY correlations are shown on the skeletal structure in red, and NOEs are depicted in blue. The inter-residue NOEs between adjacent amide protons (NH—NH) and adjacent amide and alpha protons (NH-αH) that were used to help determine connectivity are highlighted below the compound structure.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### 1. Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “carrier” as used herein refers to any pharmaceutically acceptable solvent of agents that will allow a therapeutic composition to be administered to the subject. A “carrier” as used herein, therefore, refers to such solvent as, but not limited to, water, saline, physiological saline, oil-water emulsions, gels, or any other solvent or combination of solvents and compounds known to one of skill in the art that is pharmaceutically and physiologically acceptable to the recipient human or animal. The term “pharmaceutically acceptable” as used herein refers to a compound or composition that will not impair the physiology of the recipient human or animal to the extent that the viability of the recipient is compromised. For example, “pharmaceutically acceptable” may refer to a compound or composition that does not substantially produce adverse reactions, e.g., toxic, allergic, or immunological reactions, when administered to a subject.

The term “effective amount” or “therapeutically effective amount” refers to an amount sufficient to effect beneficial or desirable biological and/or clinical results.

As used herein, the terms “subject” and “patient” are used interchangeably herein and refer to both human and nonhuman animals. The term “nonhuman animals” includes all vertebrates, e.g., mammals and non-mammals, such as nonhuman primates, sheep, dogs, cats, horses, cows, chickens, amphibians, reptiles, and the like. In some embodiments, the subject is a human. In some embodiments, the subject is a human. In particular embodiments, the subject may be male. In other embodiments, the subject may be female. In some embodiments, the subject is suffering from a bacterial infection.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. Unless otherwise defined, all technical terms used herein have the same meaning as commonly understood by one of ordinary skill in the art to which this disclosure belongs.

### 2. Methods

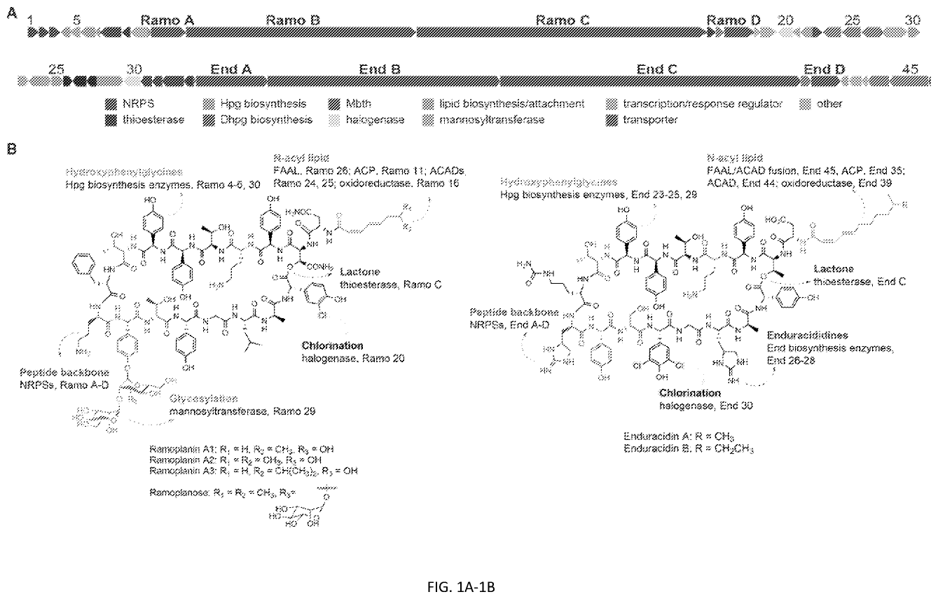

The present disclosure is based in part on findings by the inventors using a genome mining approach that has identified identify new ramoplanin family producers. The ramoplanins are an exciting family of first-generation natural products that possess excellent in vitro activity against a wide range of Gram-positive bacteria. The family is composed of nonribosomally biosynthesized lipodepsipeptides that fall into two subclasses based on structure, the ramoplanins and the enduracidins (FIG. 1).

Ramoplanins, first isolated in 1984 by fermentation of Actinoplanes (ATCC 3307) are a mixture of six lipoglycodepsipeptides of which factor A2 is most abundant, though all isomers possess similar antibiotic activities. The enduracidins A and B, lipodepsipeptides produced by Streptomyces fungicidicus B5477, are not glycosylated and contain longer N-terminal fatty acyl tails yet exhibit similar activity as ramoplanin. This antibiotic activity results from inhibition of bacterial cell wall biosynthesis. Ramoplanins and enduracidins capture the peptidoglycan (PG) biosynthesis intermediate Lipid II, the substrate for transglycosylase and transpeptidase enzymes. Sequestering this late-stage intermediate prevents formation of the mature, fully crosslinked peptidoglycan, resulting in a mechanically weakened cell wall and bacterial death due to osmotic lysis. In addition to interruption of PG biosynthesis, it has been reported that exposure of S. aureus to bactericidal concentrations of ramoplanin A2 results in membrane depolarization, suggesting a complementary mode of action through disruption of lipid membrane integrity.

Ramoplanin A2 gained initial interest for treatment of Gram-positive bacterial infections that are resistant to antibiotics such as glycopeptides, macrolides, and penicillins.^{9,12-15} It has excellent in vitro activity with MICs ranging from 0.125-2 μg/mL. However, this first-generation natural product would benefit from improvements because it is not orally absorbed, is mild to moderately hemolytic when delivered intravenously, and its macrolactone is susceptible to hydrolysis when administered by intraperitoneal injection.¹⁶ Enduracidins A and B have a similar activity profiles, but exhibit reduced solubility and have been approved only for use outside of the United States as a growth-promoting feed additive for livestock.

Despite minor limitations, ramoplanin was recently FDA approved for the treatment of Clostridium difficile colonic infections (CDI) and associated diarrhea. Oral delivery of ramoplanin achieves high colonic concentrations (>300 μg/mL), which far exceeds MICs determined in vitro against vancomycin-susceptible and vancomycin-resistant C. difficile strains (0.25-0.50 μg/mL). As such, ramoplanin remains a promising antibacterial agent warranting further development to broaden its therapeutic potential.

One underexplored avenue to develop second generation ramoplanin family members is to identify naturally produced congeners that may possess favorable structural diversities or allow for biosynthetic manipulations. In the case of glycopeptides, the development of second generation therapeutics may be promoted by identifying organisms giving rise to different core scaffolds and peripheral modifications such as acylation, glycosylation, and methylation may provide insight into mode of action and be used to prioritize semisynthetic derivatization. For example, that strains besides Actinoplanes and S. fungicidicus may harbor biosynthetic machinery for ramoplanin congener production. The identification of novel producing organisms may expand this important antibiotic class. Towards this end, presented herein is a systematic method for uncovering ramoplanin-like biosynthetic gene clusters (BGCs) within sequenced bacterial genomes.

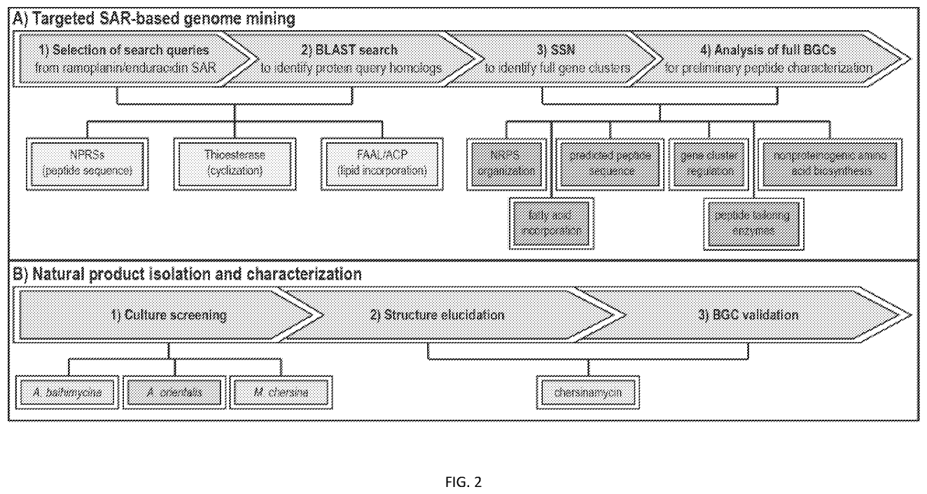

As described herein, functionally important regions within the ramoplanin and enduracidin non-ribosomal peptide synthetases (NRPS) were identified, and associated BGC standalone enzymes were used to develop a suite of key sequence probes for genome mining.^{15,16,29-38} Using these structure-activity-relationship (SAR)-informed protein sequences as search queries, a workflow that identified bacterial strains containing new lipodepsipeptide BGCs was developed. One potential workflow is shown in FIG. 2. This workflow allowed for the discovery of complete biosynthetic pathways for a ramoplanin family antibiotic in five new bacterial strains. Four of these five strains are host producers of either enediyne or glycopeptide antibiotics. One of these representative strains, the dynemicin producer Micromonospora chersina DSM 44154, was found to produce a ramoplanin congener, which was termed chersinamycin (FIG. 2B). The isolation, structure elucidation, antimicrobial activity, and validation of the BGC function using CRISPR-Cas9 gene editing is additionally described herein. These findings provide the foundation to further broaden our understanding of structure-function relationships among the ramoplanin family, to decode the molecular logic of ramoplanin biosynthesis, and to lay the foundation for the production of improved second generation ramoplanin analogs through mutasynthesis and metabolic engineering.

In one aspect, provided herein are methods for selecting a source organism of an antibiotic agent. In some embodiments, the method comprises identifying a plurality of functionally significant structural motifs within at least one parent antibiotic agent. The term “parent antibiotic agent” as used herein refers to an already known antibiotic agent from which information regarding functionally significant structural motifs is obtained. For example, for identification of novel ramoplanin congeners and/or novel sources for ramoplanin and congeners thereof, ramoplanin (e.g. ramoplanin A2) may be used as the parent antibiotic agent. In some embodiments, ramoplanin and enduracidin are used as the parent antibiotic agent.

The term “functionally significant structural motif” as used herein may refer to a protein. For example, the term “functionally significant structural motif” may refer to a protein that is important for antimicrobial activity of the parent antibiotic agent. Alternatively, the term “functionally significant structural motif” may refer to a region of a protein (e.g. a domain, a subdomain, etc.) that is important for a given function. For example, a functionally significant structural motif may be a protein or a region of a protein (e.g. protein domain) important for the antimicrobial activity of an antibiotic agent. For example, the functionally significant structural motif may be non-ribosomal peptide synthetase (NRPS) or a domain or subdomain of a non-ribosomal peptide synthetase (NRPS). Within bacteria, non-ribosomal peptide synthetases are multi-modular enzymes which catalyze the synthesis of highly diverse natural products. For example, NRPSs may catalyze the synthesis of many metabolites, including lipodepsipeptides.

In some instances, NRPSs comprise, from N-terminus to C-terminus, an initiation module (also known as a starter module or a starting module), an elongation or extending module, and a termination or releasing module. Each module may comprise multiple domains. For example, the elongation module contains three core domains. These domains are the condensation domain (C domain), the adenylation domain (A domain), and the peptidyl carrier protein (PCP) domain, which is also known as the thiolation domain (T domain). Other domains present in an NRPS may include a formylation (F) domain, a cyclization (Cy) domain, an oxidation (Ox) domain, a reduction (Red) domain, an epimerization (E) domain, an N-methylation (NMT) domain, a termination (TE) domain, a thioesterase domain, and/or an X domain. In some embodiments, a domain may have two or more functions. For example, a domain may be a dual epimerization/condensation domain.

In some embodiments, a functionally significant structural motif comprises an NRPS. In some embodiments, a functionally significant structural motif comprises any suitable domain of an NRPS. For example, a functionally significant structural motif may comprise a suitable domain for an initiation module of an NRPS. As another example, a functionally significant structural motif may comprise a suitable domain from an elongation module of an NRPS. As another example, a functionally significant structural motif may comprise a suitable domain from a termination module for an NRPS. In some embodiments, a functionally significant structural motif comprises a condensation domain (C domain), an adenylation domain (A domain), a peptidyl carrier protein (PCP) domain, a formylation (F) domain, a cyclization (Cy) domain, an oxidation (Ox) domain, a reduction (Red) domain, an epimerization (E) domain, an N-methylation (NMT) domain, a termination (TE) domain, a thioesterase domain, an X domain, and/or a dual epimerization/condensation domain of an NRPS.

The NRPS may be any member of the NRPS gene family. In some embodiments, the NRPS is selected from NRPS A, NRPS B, NRPS C, or NRPS D.

Alternatively or in addition, in some embodiments the functionally significant structural motif comprises a motif other than the NRPSs or NRPS domains described above. For example, the functionally significant structural motif may comprise a domain essential for other functions that contribute to antimicrobial activity of an antibiotic agent. For example, ramoplanins and enduracidins share genes that encode enzymes for fatty acid activation and lipoinitiation. These modifications are essential for bacterial membrane binding and antimicrobial activity. It is likely that these fatty acids originate from primary metabolism and are activated as free fatty acids. This is supported by the observation that an acyl carrier protein (ACP) and a fatty acid adenylate forming ligase (FAAL) appear in both BGCs. Accordingly, in some embodiments the functionally significant structural motif may comprise an acyl carrier protein or a domain thereof. In some embodiments, the functionally significant structural motif may comprise a fatty acid adenylate forming ligase or a domain thereof.

In some embodiments, the plurality of functionally significant structural motifs comprise a nonribosomal peptide synthetase (e.g. NRPS A, NRPS B, NRPS C, NRPS D) or a domain thereof, a fatty acid adenylate forming ligase (FAAL) or a domain thereof, and/or an acyl carrier protein (ACP) or a domain thereof. In some embodiments, the plurality of significant structural motifs comprises at least two significant structural motifs. For example, at least two, at least three, at least four, at least five, at least six, or seven or more significant structural motifs may be identified. In some embodiments, the plurality of functionally significant structural motifs comprise each of NRPS A or a domain thereof, NRPS B or a domain thereof, NRPS C or a domain thereof, NRPS D or a domain thereof, a fatty acid adenylate forming ligase (FAAL) or a domain thereof, and an acyl carrier protein (ACP) or a domain thereof.

In some embodiments, the functionally significant structural motifs are present in one parent antibiotic agent. In some embodiments, the functionally significant structural motifs are present in (e.g. shared between) at least two parent antibiotic agents. In some embodiments, the parent antibiotic agent may be a lipodepsipeptide antibiotic agent. For example, the parent lipodepsipeptide antibiotic agent may be a ramoplanin family antibiotic agent, such as ramoplanin A1, A2, A3, or enduracidin. Ramoplanin A2 is the most abundant ramoplanin family isoform, and is referred to herein as “ramoplanin”. In some embodiments, the plurality of functionally significant structural motifs are shared between ramoplanin and enduracidin.

In some embodiments, a functionally significant structural motifs may be selected based upon experimental validation of the importance of the structural motif. In some embodiments, a functionally significant structural motifs may be selected based upon existing structure-activity-relationship studies establishing the importance of the structural motif In some embodiments, the method further comprises selecting a plurality of probes.

The number of probes used will equal the number of functionally significant structural motifs identified. For example, if three functionally significant structural motifs are identified, three probes will be selected. In some embodiments, each probe comprises a nucleotide sequence encoding an identified functionally significant structural motif or an amino acid sequence of an identified functionally significant structural motif. For example, a probe for an NRPS may comprise the amino acid sequence of the NRPS. As another example, a probe for an NRPS domain may comprise the amino acid sequence of the NRPS domain. As yet another example, a probe for an NRPS may comprise a nucleotide sequence encoding the NRPS. As yet another example, a probe for an NRPS domain may comprise a nucleotide sequence encoding the NRPS domain.

In some embodiments, the method further comprises identifying homologous proteins having at least 50% sequence identity to at least one probe or to the functionally significant structural motif encoded by at least one probe. As used herein, the term “homologous proteins” refers to proteins having at least 50% sequence identity to at least one probe or to the functionally significant structural motif encoded by at least one probe. For example, homologous proteins having at least 50%, at least 55%, at least 60%, at least 65%, at least 70%, at least 75%, at least 80%, at least 85%, at least 90%, or at least 95% identity to at least one probe or to the functionally significant structural motif encoded by at least one probe may be identified. Identification of homologous proteins may be performed using a program or algorithm designed to perform sequence alignments. For example, identification of homologous proteins may be performed using a computer, wherein the computer executes a program designed to perform sequence alignments. Such programs include, for example, the NCBI protein blast program, although other programs may also be used.

In some embodiments, the method further comprises selecting a source organism when the source organism comprises at least three homologous proteins. For example, the method may comprise selecting a source organism when the source organism comprises at least three homologous proteins having at least 50% sequence identity to at least one probe or to the functionally significant structural motif encoded by the at least one probe. In some embodiments, the method comprises selecting a source organism when the source organism comprises at least four homologous proteins. Selected organisms represent a potential source for an antibiotic agent, such as a congener of the parent antibiotic agent. In some embodiments, the program or algorithm designed to perform sequence alignments also provides the user of the program with the source organism. In such embodiments, identification of homologous proteins and subsequent selection of a source organism may be performed using a computer, wherein the computer executes a program designed to perform sequence alignments and identify the source organisms. Such programs include, for example, the NCBI protein Blast program, although other programs may also be used.

In some embodiments, the method further comprises determining whether the homologous proteins (e.g. the at least three homologous proteins present in the selected source organism) form a biosynthetic gene cluster. Determination of whether the homologous proteins form a biosynthetic gene cluster may comprise obtaining whole genome sequences for each selected source organism. The whole genome sequence may be obtained from a sequence database. In other embodiments, the whole genome sequence may be obtained through sequencing methods.

In some embodiments, the method further comprises assembling a sequence similarity network (SSN) comprising each whole genome sequence and determining whether a biosynthetic gene cluster is present within the sequence similarity network. As used herein, the term “sequence similarity network” refers to a visual representation of relationships among proteins. For example, a SSN may visualize relationships among proteins and allow for identification of gene clusters (e.g. biosynthetic gene clusters) that play a role in production of an antibiotic agent within multiple source organisms. The SSN may be generated by determining the similarity of sequences (e.g. the similarity of each pair of whole genome sequences). Next, the sequences may be filtered into clusters based upon a similarity threshold value. This threshold value is defined by the user. Multiple thresholds may be used in order to generate several SSNs, which may be compared to identify biosynthetic gene clusters present across multiple similarity thresholds. In some embodiments, a SSN may be assembled using algorithms or tools available online. Suitable tools include, for example, the EFI-Enzyme Similarity Tool, although other tools or algorithms may also be used to generate the SSN.

In some embodiments, the method further comprises culturing at least one selected source organism to produce the antibiotic agent, and isolating the antibiotic agent from culture. In some embodiments, the at least one selected source organism is determined to have a biosynthetic gene cluster that facilitates production of lipodepsipeptides (e.g. lipodepsipeptide antibiotic agents). Any suitable culture conditions may be sued to facilitate production of the antibiotic agent. The culture conditions may vary depending on the source organism selected. In general, culture conditions provide a suitable temperature and nutrients (e.g. in a culture media) to promote health of the organism and facilitate production of the desired antibiotic agent.

The method may further comprise isolating the antibiotic agent. The method may further comprise purifying the antibiotic agent (e.g. further removing unwanted contaminants from the agent, resulting in a substantially pure antibiotic). In some embodiments, the antibiotic agent produced is a lipodepsipeptide antibiotic agent. For example, the antibiotic agent may be a ramoplanin congener.

In some aspects, provided herein are methods of producing an antibiotic agent. The methods comprise selecting a source organism if an antibiotic agent, using a method as described above. The methods further comprise culturing at least one selected source organism to produce the antibiotic agent as described above. The methods may further comprise isolating the antibiotic agent, and optionally purifying the antibiotic agent.

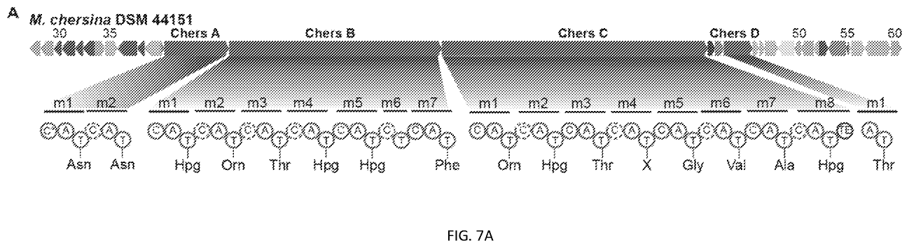

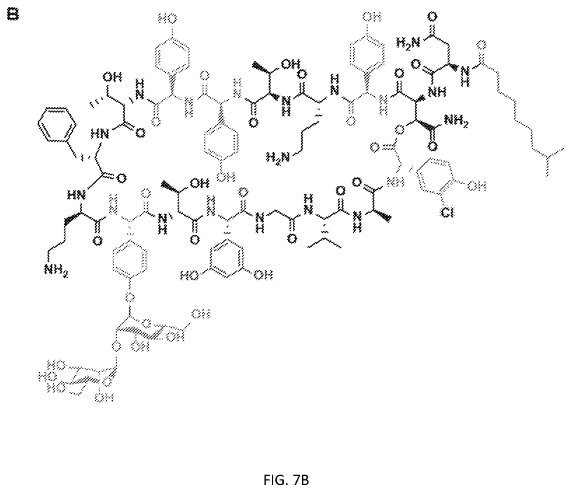

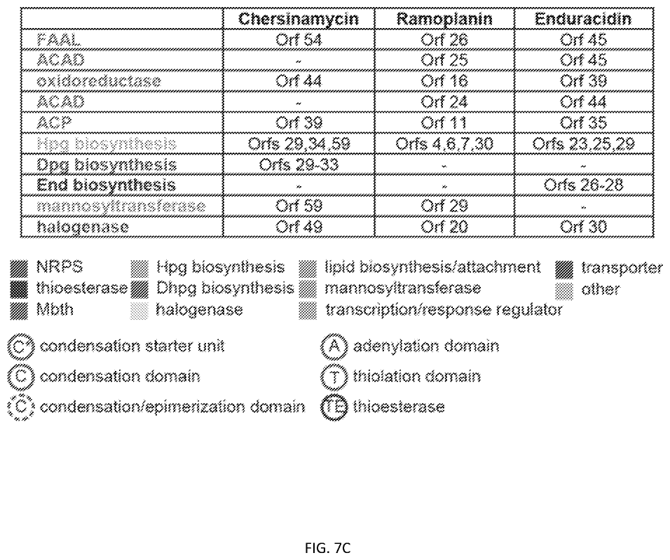

In some embodiments, the antibiotic agent produced (and optionally isolated and purified) by a method as described herein is a lipodepsipeptide antibiotic agent. For example, in some embodiments the antibiotic agent produced is a ramoplanin congener. In some embodiments, the antibiotic agent is the ramoplanin congener chersinamycin, the structure of which is shown in FIG. 7B.

In some aspects, provided herein are lipodepsipeptide antibiotic congeners for use in a method of treating bacterial infection in subject. In some embodiments, provided herein is a ramoplanin congener for use in a method of treating bacterial infection in a subject. The congener (e.g. ramoplanin congener) may be obtained using a method as described herein. In some embodiments, the congener is chersinamycin. The method may comprise providing the antibiotic agent to the subject. In some embodiments, the antibiotic agent may be formulated into a suitable pharmaceutical composition for use in a subject. For example, the agent may be formulated into a suitable pharmaceutical composition comprising one or more carriers for delivery to a subject to treat a bacterial infection. Selection of the appropriate carriers will depend on the mode of administration.

Contemplated routes of administration include oral, rectal, nasal, topical (including transdermal, buccal and sublingual), vaginal, parenteral (including subcutaneous, intramuscular, intravenous and intradermal) and pulmonary administration. In some embodiments, the composition or compositions are conveniently presented in unit dosage form and are prepared by any method known in the art of pharmacy. Such methods include the step of bringing into association the active ingredient (e.g. the antibiotic agent) with the carrier. In general, the formulations are prepared by uniformly and intimately bringing into association (e.g., mixing) the active ingredient (e.g. the antibiotic agent) with liquid carriers or finely divided solid carriers or both, and then if necessary shaping the product.

Formulations of the present disclosure suitable for oral administration may be presented as discrete units such as capsules, cachets or tablets, wherein each preferably contains a predetermined amount of the one or more therapeutic agents as a powder or granules; as a solution or suspension in an aqueous or non-aqueous liquid; or as an oil-in-water liquid emulsion or a water-in-oil liquid emulsion. In other embodiments, the composition is presented as a bolus, electuary, or paste, etc. Preferred unit dosage formulations are those containing a daily dose or unit, daily sub dose, or an appropriate fraction thereof, of an agent.

It should be understood that in addition to the ingredients particularly mentioned above, the compositions may include other agents conventional in the art having regard to the route of administration in question. For example, compositions suitable for oral administration may include such further agents as sweeteners, thickeners and flavoring agents. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, preservatives, and stabilizers, etc.

Various delivery systems are known and can be used to administer compositions described herein, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the compositions of the disclosure locally to the area in need of treatment; this may be achieved by, for example, and not by way of limitation, local infusion during surgery, injection, or by means of a catheter.

Therapeutic amounts (e.g. amounts of the antibiotic agent) are empirically determined and vary with the pathology being treated, the subject being treated and the efficacy and toxicity of the agent. It is understood that therapeutically effective amounts vary based upon factors including the age, gender, and weight of the subject, among others. It also is intended that the compositions and methods of this disclosure be co-administered with other suitable compositions and therapies.

In some embodiments, the bacterial infection is an infection associated with one or more Gram-positive bacterium. In some embodiments, the Gram-positive bacterium is a species belonging to the Enterococcus, Macrococcus, Staphylococcus, Streptococcus, Actinomycetes, Bacillus, Clostridium, Corynebacterium, Ersipeloxhtirx, Listeria, Mycobacterium, Nocardia, Rhodococcus, or Streptomyces family. In some embodiments, the gram-positive bacterium is pathogenic (e.g. causes sickness) in humans. Any suitable pathogenic gran-positive bacteria may be the cause of an infection that may be treated with an antibiotic agent described herein.

In some embodiments, the Gram-positive bacterium is a Staphylococcus species selected from Staphylococcus aureus, Staphylococcus epidermis, Staphylococcus saprophyticus, Staphylococcus haemolyticus, Staphylococcus hominis, and Staphylococcus lugdunensis. In some embodiments, the Gram-positive bacterium is a Streptococcus species selected from Streptococcus pneumoniae, Streptococcus pyrogenes, and Streptococcus agalactiae. In some embodiments, the gram-positive bacterium is an Enterococcus species, such as Enterococcus faecium or Enterococcus faecalis. In some embodiments, the Gram-positive bacterium is a Bacillus species selected from Bacillus anthraces and Bacillus cereus. In some embodiments, the Gram-positive bacterium is a species of Clostridium selected from Clostridium botulinum, Clostridium perfringens, Clostridium difficile, and Clostridium tetani.

In some embodiments, the Gram-positive bacterium is Listeria monocytogenes. In some embodiments, the Gram-positive bacterium is Corynebacterium diptheria. In some embodiments, the bacterial infection is associated with S. aureus, C. difficile, E. faecium, or E. faecalis infection. Infection with the gram-positive bacterium may cause any number of symptoms in a subject. Treating the infection with an antibiotic agent as described herein may reduce or improve the one or more symptoms.

### 3. Examples

### Example 1

Targeted Genome Mining discovery of the Ramoplanin Congener Chersinamycin from the Dynemicin-Producer Micromonospora chersina DSM 44154

### Overview:

Ramoplanin is a lipoglycodepsipeptide antibiotic that is highly effective against Gram-positive pathogens, including several strains that are resistant to first line antibiotics such as methicillin and vancomycin. Though it has achieved success in early clinical trials and is a hopeful candidate for the treatment of Clostridium difficile infections, the full therapeutic potential of ramoplanin is somewhat hindered due to issues with stability and tolerability upon intravenous injection. Analogs with more desirable biological properties are needed but difficult to access synthetically due to its complex structure.

Herein, a targeted genome mining approach was developed to uncover natural sources of new ramoplanin family compounds to access new scaffolds and afford opportunities for biosynthetic manipulation and analog development. By selecting results of structure-function studies of ramoplanin and enduracidin to guide the search, the approach described herein allowed for the rapid identification of five new lipodepsipeptide biosynthetic gene clusters of the ramoplanin/enduracidin family. These gene clusters were discovered in well-characterized natural product-producing organisms such as glycopeptide antibiotic producers Amycolatopsis orientalis and Amycolatopsis balhimycina and enediyne anti-cancer compound producer Micromonospora chersina.

In silico analyses of the biosynthetic gene clusters have identified new scaffolds for investigation. Growth and extraction of strain M. chersina led to the isolation and characterization of chersinamycin, a new lipoglycodepsipeptide with potent antimicrobial activity against Gram-positive bacteria. The chersinamycin gene cluster was confirmed through CRISPR-Cas9-mediated knockout of nonproteinogenic amino acid biosynthesis genes within the cluster. As it is produced in a genetically tractable organism, the discovery of chersinamycin provides exciting opportunities for investigation into the biosynthetic machinery of peptide production, as well as opportunity for the biosynthesis and semisynthesis of new antibiotics, thus allowing for further development of this potent peptide class and expansion of the human arsenal of antibiotics to combat antibiotic crisis.

### Results:

BGCs of ramoplanin and enduracidin share conserved sequences linked to functionally important structural features. The methods of searching for new ramoplanin family lipodepsipeptide gene clusters described herein began with genome mining for key biosynthetic proteins, a process that was unique in that it was guided by results from structure-function studies of ramoplanins and enduracidins. There are several general shared structural features of these antibiotics that are critically important for their activity: (1) Conserved amino acid type and stereochemistry within the 17-residue depsipeptide, which influences the overall peptide receptor-like conformation, promotes antibiotic dimerization^{34,40,50} and facilitates binding to its lipid II target^{9,15,37,38} (2) Conformational constraint imparted by the 49-atom macrocycle; and (3) N-terminal acylation, which promotes bacterial membrane association and influences its amphipathic C2 symmetrical dimeric conformation that is adopted upon membrane binding.

Common to the ramoplanin and enduracidin BGCs are four non-ribosomal peptide synthetases (NRPSs) termed Ramo/End A-D (FIG. 1A), which encode enzymes responsible for assembly line synthesis of these 17-residue peptides, including 12 nonstandard amino acids and seven with a D-amino acid configuration. Three large NRPS ORFs (A, B, C) appear to be organized in accordance with the collinearity rule of modular construction of NRPS condensation, adenylation, and thiolation domains. The exception is ramoD/endD, which encodes a standalone adenylation/thiolation di-domain enzyme that is predicted to work in trans with the NRPS B dual condensation/epimerization (C/E) domain to introduce D-allo-Thr8 within the linear peptide sequence.

Within the primary sequences of ramoplanin and enduracidin, there are several conserved residues that have been strongly linked to lipid II binding affinity and antibiotic activity. Boger and colleagues elegantly employed total solution-phase synthesis to perform an alanine scan of ramoplanin A2 residues 3-13, 15, and 17 within [Dap2]-ramoplanin A2 aglycon, a hydrolytically stable ramoplanin aglycon analog. When compared to ramoplanin A1-A3 complex (MIC=0.19 μg/mL), ramoplanin A2 aglycon (MIC=0.11 μg/mL), and [Dap2]-ramoplanin aglycon (MIC=0.07 μg/mL), alanine substitution of these 12 positions resulted in MIC increases over the parent antibiotics ranging from 1.3 to 540-fold (FIG. 1B). Three residues exhibited markedly increased MICs: D-allo-Thr5 (74-fold), D-Hpg7 (53-fold) and D-Orn10 (540-fold). Residues 5 and 7 lie within the D-allo-Thr5-Hpg6-D-Hpg7-D-allo-Thr8 sequence that is conserved with enduracidins, and residue 10 is functionally conserved in enduracidins as D-enduracididine (End). Subsequently, Boger, Walker, and coworkers determined the effect of alanine substitution on lipid II binding and penicillin binding protein inhibition using a [Dap2]-ramoplanin A2 amide scaffold that was modified by the inclusion of single alanines along positions 3-12. The introduction of Ala residues increased Kd values ranging from 378-8700 nM, with positions 4,8, and 10-12 exhibiting>100-fold increased Kd. Analogs that exhibited the most significant changes in MIC and Kd values were considered to be functionally important and therefore likely to be conserved within a new ramoplanin/enduracidin congener. As such, these regions were carefully considered when devising the genome mining strategy described herein.

In addition, Williams and coworkers first demonstrated that hydrolysis of the macrolactone bond of ramoplanose resulted in a markedly less soluble linear peptide that lacked antimicrobial activity. Boger and coworkers showed that ramoplanin A2 activity required a 49-membered macrocycle, regardless of whether the macrocycle was linked by a lactone or lactam bond. Within Ramo C/End C NRPSs, the C-terminal thioesterase domain is responsible for installing this indispensable macrocycle and was considered a key biosynthetic sequence to be included as a genome mining search query.

Ramoplanins and enduracidins share genes that encode enzymes for fatty acid activation and lipoinitiation, the modification essential for bacterial membrane binding and antimicrobial activity. Both BGCs lack candidate ORFs encoding enzymes for de novo fatty acid biosynthesis, so it is likely that these fatty acids originate from primary metabolism and are activated as free fatty acids.^{32,47} In support of this hypothesis, an acyl carrier protein (ACP) and a fatty acid adenylate forming ligase (FAAL) appear in both BGCs. The presence of an N-terminal C^{III} condensation domain in NRPS A of both BGCs further supports a lipoinitiation mechanism involving fatty acid activation and condensation with residue 1 to form the starting N-acyl amino acid starter unit.

Although both antibiotic BGCs contain conserved acyl-CoA dehydrogenases (ACADs) and oxidoreductases that are believed to install the E,Z fatty acid double bonds, these enzymes are likely non-essential, since loss of these double bonds by hydrogenation of ramoplanin A251 or semisynthesis resulted in no significant reduction in antimicrobial activity. Similarly, mannosylation and chlorination are structural elements that have been shown to be nonessential for antibiotic activity, although mannosylation has been shown to enhance the conformational stability of ramoplanin A229, and improve solubility over enduracidin.

Collectively, these studies link membrane association, antimicrobial activity, and lipid II binding with specific structural elements shared between ramoplanin and enduracidin. By correlating functionally important architectural features with corresponding BGC-encoded enzymes that are responsible for their assembly, a set of probes for genome mining to search for ramoplanin congeners was developed herein.

Discovery of ramoplanin-like biosynthetic gene clusters by genome mining: BGC sequences of 7 SAR-guided probes from the NRPSs A-D, the acyl carrier proteins (ACP), and FAALs from the ramoplanin and enduracidin BGCs were used as initial BLASTp search queries to identify homologs from bacterial strains within the NCBI database. Protein sequence hits with >50% identity to the search queries were collected and cross-referenced to microbial strains that met the criteria of containing at least 4 homologs within its genome, regardless of ORF location. With these initial boundary conditions, 13 microbial strains were identified (Table 1).

TABLE 1
Identified bacterial strains with homologs to key ramoplanin and enduracidin biosynthesis proteins.

| Organism/Name | NRPS A | NRPS B | NRPS C | NRPS D | FAAL | ACP | Thioesterase |
|---|---|---|---|---|---|---|---|
| Streptomyces fungicidicus | R | R | R | R | R | R | R |
| ATCC 21013 (enduracidin) | | | | | | | |
| Micromonospora chersina | R | R, E | R, E | R, E | R, E | R, E | R, E |
| strain DSM 44151 | | | | | | | |
| Amycolatopsis orientalis | R, E | R, E | R, E | R, E | R, E | R, E | R, E |
| strain B-37 | | | | | | | |
| Amycolatopsis orientalis | R, E | R, E | R, E | R, E | R, E | R, E | R, E |
| DSM 40040 = KCTC 9412 | | | | | | | |
| Amycolatopsis balhimycina | R, E | R, E | R, E | R, E | R, E | R, E | R, E |
| FH 1894 strain DSM 44591 | | | | | | | |
| Streptomyces sp. TLI_053 | | R, E | R, E | R, E | R, E | R, E | R, E |
| Micromonospora sp. MH33 | | R, E | R, E | R, E | R, E | R, E | R, E |
| Amycolatopsis thailandensis | R, E | R, E | E | | R, E | R, E | R, E |
| srain JCM 16389 | | | | | | | |
| Actinomadura madurae | | R | R, E | | R, E | R | E |
| LIID-AJ290 | | | | | | | |
| Actinomadura madurae | | R | R, E | | R, E | R | E |
| strain DSM 43067 | | | | | | | |
| Streptomyces vietnamensis | E | E | | | R, E | R, E | |
| strain GIM4.0001 | | | | | | | |
| Streptomyces sp. GP55 | E | E | | | R, E | R, E | |
| Streptomyces cinnamoneus | | R, E | | | R, E | R, E | R, E |
| strain ATCC 21532 | | | | | | | |
| Streptomyces cinnamoneus | | | R, E | | R, E | R, E | R, E |
| strain DSM 41675 | | | | | | | |

Analyzed proteins are Ramo A/End A, Ramo B/End B, Ramo C/End C, Ramo D/End D, and each respective FAAL, ACP, and terminal thioesterase of NRPS C. An R indicates >50% identity to the ramoplanin homologue and E indicates >50% identity to the enduracidin homolog.

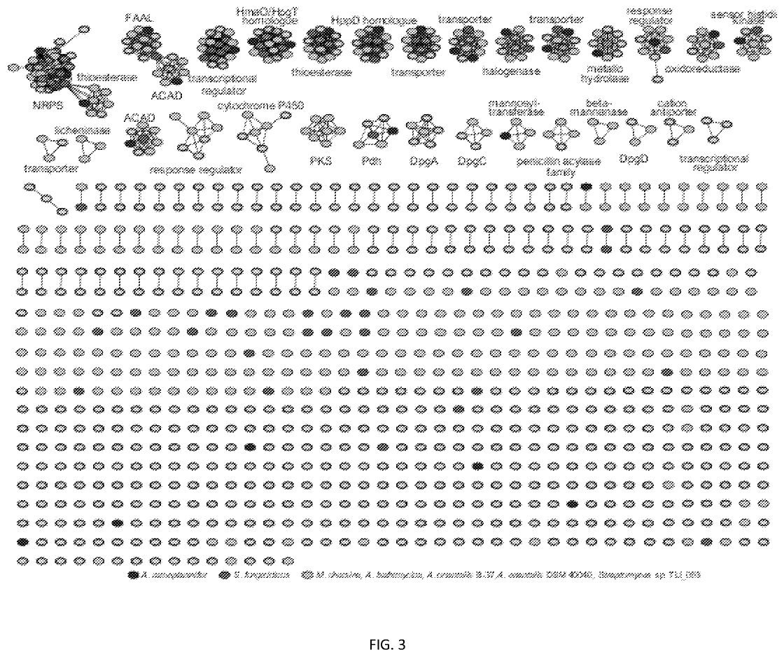

To determine if the protein homologs from the 13 strains were organized into a single BGC, the sequence analysis was expanded. Given the importance of the primary sequence encoded by the Ramo B/End B NRPS to the activity of ramoplanin and enduracidin, the translated sequences were analyzed within forty ORFs on either side of each NRPS B hit. Sequences obtained from the NCBI protein database were submitted to the EFI-Enzyme Similarity Tool for an all vs. all Blast search and assembly into a sequence similarity network (SSN) (FIG. 3).

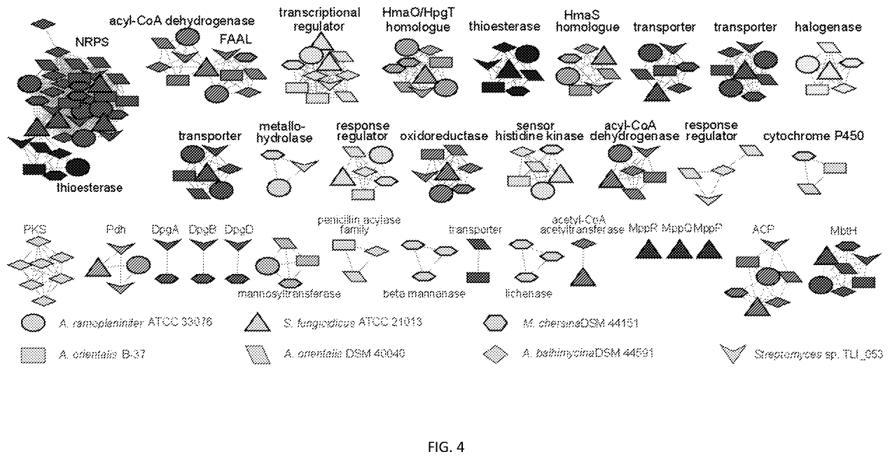

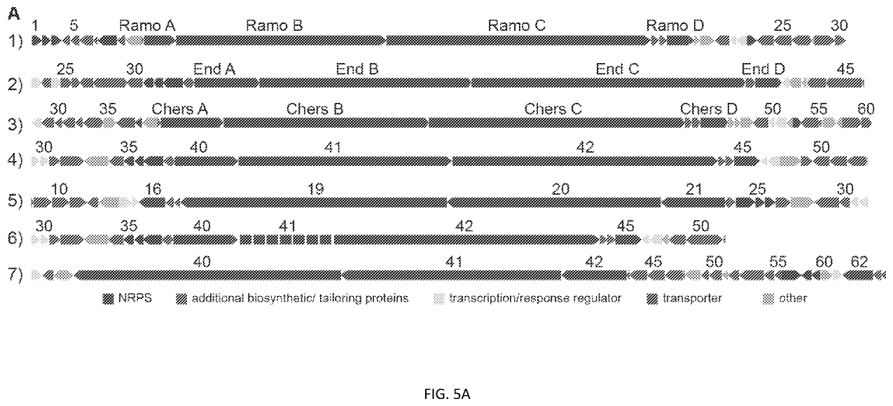

The SSN revealed clear protein clusters representing nearly all of the proteins within the defined ramoplanin and enduracidin BGCs; only five of the 24 proteins in the enduracidin BGC32 and six of the 31 proteins in the ramoplanin BGC31 are represented as isolated nodes. Though multiple proteins from each of the 13 preliminary strains were present within these clusters, five strains contained all 7 of the proteins utilized as genome mining probes localized to a single region of the genome. In addition, within the analyzed region of each of these five strains a significant number of ORFs were homologous to ramoplanin and enduracidin ORFs involved in nonproteinogenic amino acid synthesis, transcriptional regulation, and natural product transport. The strains found to encode a putative BGC for ramoplanin/enduracidin congener production include Micromonospora chersina strain DSM 44151, Amycolatopsis orientalis strain B-37, Amycolatopsis orientalis strain DSM 40040, Amycolatopsis balhimycina FH1894 strain DSM 44591, and Streptomyces sp. TLI 053 (FIG. 4). Remarkably, four of these five new BGCs reside within bacterial strains that have been cultured and extracted for previously characterized natural products, including A. orientalis DSM 40040 and A. balhimycina FH1894, which produce the glycopeptide antibiotics vancomycin and balhimycin, respectively, and M. chersina DSM 44151, which produces the enediyne antibiotic dynemicin.

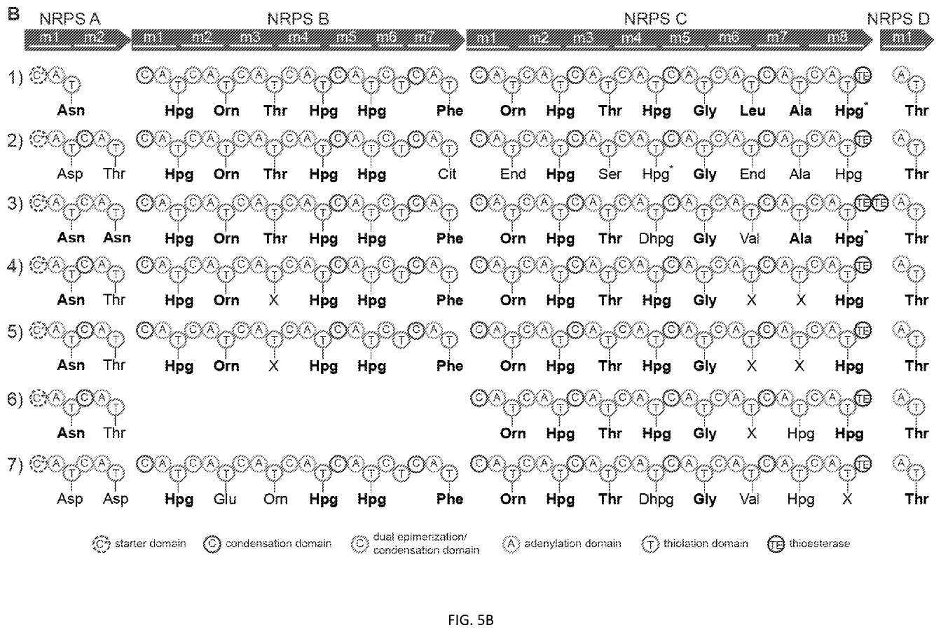

The bounds of each of the five new BGCs were determined by analyzing clustered proteins within the SSN (FIG. 4, FIG. 5A). Remarkable similarity was identified between ORFs included within the BGCs from each strain. The absence of clustered proteins not found within ramoplanin and enduracidin BGCs supports the previously defined bounds of these clusters. The gene organization and degree of conservation between each BGC likely reflects the necessity of nearly every protein in the cluster.

The SAR-guided genome mining approach allowed for the identification of five complete BGCs with strong similarity to the ramoplanin/enduracidin BGCs, suggesting that these five microorganisms contain the biosynthetic machinery to produce ramoplanin-like compounds. Manual analyses of increasingly stringent search criteria had the advantage of identifying candidates with inverted or varied organization of ORFs within the cluster, making them unable to be predicted by algorithms used by programs such as antiSMASH. This method was advantageous because it quickly allowed the selection criteria for hits to be filtered to select those most likely to belong to the desired antimicrobial class.

In silico analysis of the NRPSs: Each of the five BGCs contained four NRPSs that are predicted to incorporate 17 amino acids into the peptide (FIG. 5B). The organization of the NRPSs within each BGC was very similar to the ramoplanin and enduracidin NRPSs, including the presence of a standalone A-T domain of NRPS D, which suggests that these NRPSs also operate in trans with module 6 of each NRPS B, which contains only C and T domains. NRPS A from each new cluster contains two full modules for the incorporation of two amino acids, leaving Ramo A as a unique NRPS in which a single module is predicted to act in an iterative fashion to assemble the first two asparagine residues.

The linear peptide sequence from each cluster was predicted from the adenylation domain specificity-conferring sequences. Web-based prediction software including NRPSPredictor261 and the PKS/NRPS Analysis Web Site62 was complemented with manual sequence alignment of the ten conserved adenylation domain active site residues to account for genus-dependent sequence variation as well as a lack of predictive power for some unnatural amino acids by web-based software (Table 2, FIG. 4B).

TABLE 2
Amino acid sequence comparison of predicted peptide products from
ramoplanin family BGCs.

| | Substrate | | |
|---|---|---|---|
| | Recognition | AntiSMASH/ | Confirmed |
| Module | Sequence | NRPSPredictor2 | amino acid |
| NRPS 1 m1 | | | |
| RamoA-m1 | DLTKVGEV | L-Asn/Asn | Lipo-L-Asn¹ |
| EndA-m1 | DLTKVGHV | L-Asp/Asp | Lipo-L-Asp¹ |
| ChersA-m1 | DLTKVGEV | D-Asn/Asn | Lipo-D-Asn¹ |
| A. orientalis B-37-m1 | DLTKVGEV | L-Asn/Asn | |
| A. orientalis DSM 40040-m1 | DLTKVGEVf | L-Asn/Asn | |
| A. balhimycina-m1 | DLTKVGEV | L-Asn/Asn | |
| Streptomyces sp. TLI-053-m1 | DLTKVGHI | D-Asp/Asp | |
| NRPS 1 m2 | | | |
| RamoA-m2 | — | — | β-OH-L-Asn² |
| EndA-m2 | DFWSVGMV | L-Thr/Thr | L-Thr² |
| ChersA-m2 | DLTKVGEV | L-Asn/Asn | β-OH-L-Asn² |
| A. orientalis B-37-m2 | DFWSVGMV | L-Thr/Thr | |
| A. orientalis DSM 40040-m2 | DFWSVGMV | L-Thr/Thr | |
| A. balhimycina-m2 | DFWSVGMV | L-Thr/Thr | |
| Streptomyces sp. TLI-053-m2 | DLTKVGHI | L-Asp/Asp | |
| NRPS 2 m1 | | | |
| RamoB-m1 | DAYHLGLL | D-Hpg/Hpg | D-Hpg³ |
| EndB-m1 | DAYHLGLL | D-Hpg/Hpg | D-Hpg³ |
| Chers B-m1 | DAYHLGLL | D-Hpg/Hpg | D-Hpg³ |
| A. orientalis B-37-m1 | DAYALGLL | D-Hpg/Hpg | |
| A. orientalis DSM 40040-m1 | DAYHLGLL | D-Hpg/Hpg | |
| A. balhimycina-m1 | No sequencing data | | |
| Streptomyces sp. TLI-053-m1 | DAYHLGLL | D-Hpg/Hpg | |
| NRPS 2 m2 | | | |
| RamoB-m2 | DMDTLVSV | D-X/Tyr, Bht | D-Orn⁴ |
| EndB-m2 | DMETDGSV | D-X/Orn, Lys, Arg | D-Orn⁴ |
| Chers B-m2 | DMETDGSV | D-X/Orn, Lys, Arg | D-Orn⁴ |
| A. orientalis B-37-m2 | DMET-GSV | D-X/Orn, Lys, Arg | |
| A. orientalis DSM 40040-m2 | DMETDGSV | D-X/Orn, Lys, Arg | |
| A. balhimycina-m2 | No sequencing data | | |
| Streptomyces sp. TLI-053-m2 | DVWHFGQI | d-Glu/Glu | |
| NRPS 2 m3 | | | |
| RamoB-m3 | DFWSVGMW | D-Thr/Thr | D-allo-Thr⁶ |
| EndB-m3 | DFWSVGMV | D-Thr/Thr | D-allo-Thr⁶ |
| Chers B-m3 | DFWSVGMV | D-Thr/Thr | D-allo-Thr⁶ |
| A. orientalis B-37-m3 | DLES-GTV | D-X/Orn, Lys, Arg | |
| A. orientalis DSM 40040-m3 | DLESDGTV | D-X/Orn, Lys, Arg | |
| A. balhimycina-m3 | No sequencing data | | |
| Streptomyces sp. TLI-053-m3 | DMETLVSV | D-X/Orn, Lys, Arg | |
| NRPS 2 m4 | | | |
| RamoB-m4 | DAYHLGLL | L-Hpg/Hpg | L-Hpg⁶ |
| EndB-m4 | DAYHLGLL | L-Hpg/Hpg | L-Hpg⁶ |
| Chers B-m4 | DAYHLGLL | L-Hpg/Hpg | L-Hpg⁶ |
| A. orientalis B-37-m4 | DAY-LGLL | L-Hpg/Hpg | |
| A. orientalis DSM 40040-m3 | DAYHLGLL | L-Hpg/Hpg | |
| A. balhimycina-m4 | No sequencing data | | |
| Streptomyces sp. TLI-053-m4 | DAYHLGLL | L-Hpg Hpg | |
| NRPS 2 m5 | | | |
| RamoB-m5 | DAYHLGLL | D-Hpg/Hpg | D-Hpg⁷ |
| EndB-m5 | DAYHLGLL | D-Hpg/Hpg | D-Hpg⁷ |
| Chers B-m5 | DAYHLGLL | D-Hpg/Hpg | D-Hpg⁷ |
| A. orientalis B-37-m5 | DAYALGLL | D-Hpg/Hpg | |
| A. orientalis DSM 40040-m5 | DAYHLGLL | D-Hpg/Hpg | |
| A. balhimycina-m5 | No sequencing data | | |
| Streptomyces sp. TLI-053-m5 | DAYALGLL | D-Hpg/Hpg | |
| NRPS 2 m6 | | | |
| RamoB-m6 | No A domain | - | L-allo-Thr⁸ |
| EndB-m6 | No A domain | - | L-allo-Thr⁸ |
| Chers B-m6 | No A domain | - | L-allo-Thr⁸ |
| A. orientalis B-37-m6 | No A domain | - | |
| A. orientalis DSM 40040-m6 | No A domain | - | |
| A. balhimycina-m6 | No sequencing data | | |
| Streptomyces sp. TLI-053-m6 | No A domain | - | |
| NRPS 2 m7 | | | |
| RamoB-m7 | DAWTVAAV | L-Phe/Phe | L-Phe⁹ |
| EndB-m7 | DMEADGAV | L-hydrophillic | L-Cit⁹ |
| Chers B-m7 | DAWTVAAV | L-Phe/Phe | L-Phe⁹ |
| A. orientalis B-37-m7 | DAWTVAAV | L-Phe/Phe | |
| A. orientalis DSM 40040-m7 | DAWTVAAV | L- Phe/Phe | |
| A. balhimycina-m7 | No sequencing data | | |
| Streptomyces sp. TLI-053-m7 | DAWTVAAV | L- Phe/Phe | |
| NRPS 3 m1 | | | |
| RamoC-m1 | DMDTDGSV | D-X/unknown | D-Orn¹⁰ |
| EndC-m1 | DAETDGSV | D-X/Orn, Lys, Arg | D-End¹⁰ |
| ChersC-m1 | DMETDGSV | D-X/Orn, Lys, Arg | D-Orn¹⁰ |
| A. orientalis B-37-m1 | DMETDGSV | D-X/Orn, Lys, Arg | |
| A. orientalis DSM 40040-m1 | DMETDGSV | D-X/Orn, Lys, Arg | |
| A. balhimycina-m1 | DMETDGSV | D-X/Orn, Lys, Arg | |
| Streptomyces sp. TLI-053-m1 | DMETLVSV | D-X/Orn, Lys, Arg | |
| NRPS 3 m2 | | | |
| RamoC-m2 | DAFXLGLL | L-Hpg/Hpg | L-Hpg¹¹ |
| EndC-m2 | DAYHLGML | L-Hpg/Hpg | L-Hpg¹¹ |
| ChersC-m2 | DAYHLGLL | L-Hpg/Hpg | L-Hpg¹¹ |
| A. orientalis B-37-m2 | DAYHLGLL | L-Hpg/Hpg | |
| A. orientalis DSM 40040-m2 | DAYHLGLL | L-Hpg/Hpg | |
| A. balhimycina-m2 | DAYHLGML | L-Hpg/Hpg | |
| Streptomyces sp. TLI-053-m2 | DAYHLGLL | L-Hpg/Hpg | |
| NRPS 3 m3 | | | |
| RamoC-m3 | DFWSVGMV | D-Thr/Thr | D-allo-Thr¹² |
| EndC-m3 | DVWSVAMV | D-X/unknown | D-Ser¹² |
| ChersC-m3 | DFWSVGMV | D-Thr/Thr | D-allo-Thr¹² |
| A. orientalis B-37-m3 | DFWSVGMV | D-Thr/Thr | |
| A. orientalis DSM 40040-m3 | DFWSVGMV | D-Thr/Thr | |
| A. ba/himycina-m3 | DFWSVGMV | D-Thr/Thr | |
| Streptomyces sp. TLI-053-m3 | DFWNVGMV | D-Thr/Thr | |
| NRPS 3 m4 | | | |
| RamoC-m4 | DAYHLGLL | L-Hpg/Hpg | L-Hpg¹³ |
| EndC-m4 | DAYHLGLL | L-Hpg/Hpg | L-DiCIHpg¹³ |
| ChersC-m4 | DALSLGTV | L-X/Phe, Trp, Phg, Tyr, Bht | L-Dpg¹³ |
| A. orientalis B-37-m4 | DAYHLGLL | L-Hpg/Hpg | |
| A. orientalis DSM 40040-m4 | DAYHLGLL | L-Hpg/Hpg | |
| A. balhimycina-m4 | DAFHLGLL | L-Hpg/Hpg | |
| Streptomyces sp. TLI-053-m4 | DALSLGTV | L-X/Gly, Ala, Val, Leu, | |
| | | Ile, Abu, Iva | |
| NRPS 3 m5 | | | |
| RamoC-m5 | DILQLGLV | Gly/Gly | Gly¹⁴ |
| EndC-m5 | DILQLGLV | Gly/Gly | Gly¹⁴ |
| ChersC-m5 | DILQLGLV | Gly/Gly | Gly¹⁴ |
| A. orientalis B-37-m5 | DILQVGLV | Gly/Gly | |
| A. orientalis DSM 40040-m5 | DILQLGLV | Gly/Gly | |
| A. balhimycina-m5 | DILQLGLV | Gly/Gly | |
| Streptomyces sp. TLI-053-m5 | DILQXXLV | Gly/Gly | |
| NRPS 3 m6 | | | |
| RamoC-m6 | DAFFYGAT | L-lle/lle | L-Leu¹⁶ |
| EndC-m6 | DAETDGSV | l- X/Orn, Lys, Arg | L-End¹⁶ |
| ChersC-m6 | DAFWLGGT | L-Val/Val | L-Val¹⁶ |
| A. orientalis B-37-m6 | DAMLVGAV | L-X/Val, Leu, Ile, Abu, Iva | |
| A. orientalis DSM 40040-m6 | DAMLVGAL | L-X/Val, Leu, Ile, Abu, Iva | |
| A. balhimycina-m6 | DAMLVGAV | L-X/Val, Leu, Ile, Abu, Iva | |
| Streptomyces sp. TLI-053-m6 | DALWLGGT | L-Val/Val | |
| NRPS 3 m7 | | | |
| RamoC-m7 | DVFSVAIL | D-Ala | D-Ala¹⁶ |
| EndC-m7 | DIFQLALV | D-X/Gly, Ala | D-Ala¹⁶ |
| ChersC-m7 | DVFSVAIV | D-Ala | D-Ala¹⁶ |
| A. orientalis B-37-m7 | DMET-GTV | D-hydrophillic | |
| A. orientalis DSM 40040-m7 | DMETDGTV | D-hydrophillic | |
| A. balhimycina-m7 | DAYHLGLL | D-Hpg | |
| Streptomyces sp. TLI-053-m7 | DAYHLGLL | D-Hpg | |
| NRPS 3 m8 | | | |
| RamoC-m8 | DAYHLGLL | L-Hpg/Hpg | L-CIHpg¹⁷ |
| EndC-m8 | DAYHLGLL | L-Hpg/Hpg | L-Hpg¹⁷ |
| ChersC-m8 | DAYHLGML | L-Hpg/Hpg | L-CIHpg¹⁷ |
| A. orientalis B-37-m8 | DAYHLGLL | L-Hpg/Hpg | |
| A. orientalis DSM 40040-m8 | DAYHLGLL | L-Hpg/Hpg | |
| A. balhimycina-m8 | DAYHLGLL | L-Hpg/Hpg | |
| Streptomyces sp. TLI-053-m8 | DALILGTV | L-X/Gly, Ala, Val, Leu, | |
| | | Ile, Abu, Iva | |
| NRPS 4 | | | |
| RamoD | DFWNIGMV | L-Thr/Thr | L-allo-Thr⁸ |
| EndD | DFWSVGMV | L-Thr/Thr | L-allo-Thr⁸ |
| ChersD | DFWNIGMV | L-Thr/Thr | L-allo-Thr⁸ |
| A. orientalis B-37 | DFWSIGMV | L-Thr/Thr | |
| A. orientalis DSM 40040 | DFWSIGMV | L-Thr/Thr | |
| A. balhimycina | DFWSVGMV | L-Thr/Thr | |
| Streptomyces sp. TLI-053 | DFWSVGMV | L-Thr/Thr | |

The eight adenylation domain specificity-conferring sequences were identified and predictions for the encoded amino acid are based on antiSMASH consensus and NRPSPredictor2. D- or L-stereochemistry is predicted based on the presence of ^{L}CL or E/C domains following the adenylation domain indicated.

For each organism, the NRPS-encoded primary sequences clearly predicted that all were likely ramoplanin congeners, yet each predicted sequence was unique and not identical to enduracidin or ramoplanin. Despite these differences, the NRPSs exhibited nearly identical conservation of five “hot spot” residues (Orn4, Thr8, Orn10, Hpg11, and Thr12) that had been identified in ramoplanin as having the highest contribution to lipid II binding and antimicrobial activity and that are functionally conserved in enduracidin. The only exception is residue 4 of the product encoded by the Streptomyces sp. TLI_053 NRPS, which predicts the ornithine is shifted to residue position 5 (FIG. 5B).

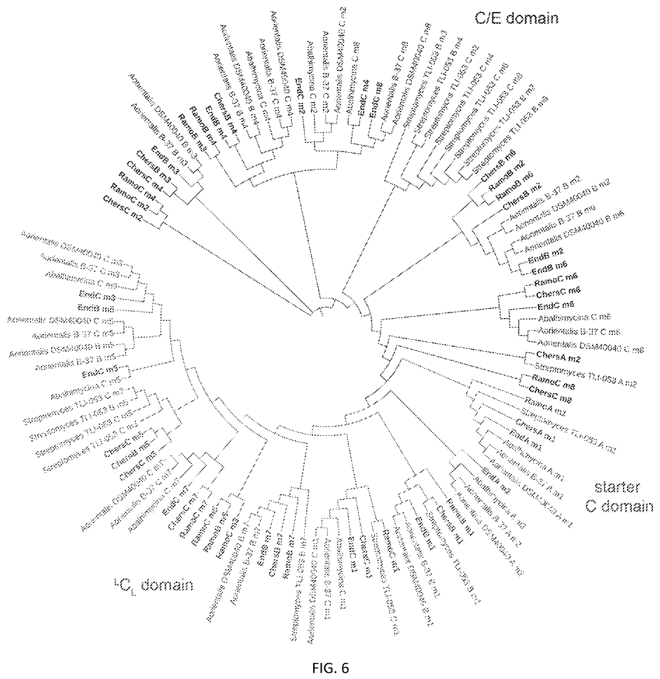

Condensation domain sequences within the NRPSs were also examined using antiSMASH predictions and manual sequence alignment to identify C-domain subtypes (FIG. 6). Each of the five organisms share a conserved starter condensation domain (CIII) as the first domain of NRPS A for fatty acid incorporation at the N-terminal residue, consistent with the presence of a FAAL and ACP within the BGC and necessity of N-acylation for activity of ramoplanin and enduracidin. The order of classical LCL and dual C/E domains, responsible for incorporating L- and D-amino acids, respectively, exactly matches those found in the ramoplanin and enduracidin NRPSs within every module from the five new clusters (with D-amino acids in positions 3, 4, 5, 7, 8, 10, 12, and 16), with a single exception at NRPS A-module 2 of M. chersina and Streptomyces sp. TLI_053 NRPS A (FIG. 5B and FIG. 6).

Screening new bacterial strains for ramoplanin congener production: In an effort to identify and isolate new ramoplanin congeners, the three strains M. chersina DSM 44151, A. orientalis DSM 40040, and A. balhimycina FH 1894 strain DSM 44591 were examined for production of ramoplanin-like molecules. Initial media formulations screened included the optimized media for ramoplanin and enduracidin production, as well as the media optimized for production of each strain's characterized natural product. Following incubation at various time intervals, cultures were extracted and screened by MALDI-TOF for a peptide within a mass range chosen based on bioinformatic predictions.

Although ramoplanin-like molecules were not observed to be produced by fermentation of either A. orientalis DSM 40040 or A. balhimycina, fermentation of M. chersina for 12 days in dynemicin production medium H881 resulted in the production of a compound with a mass of 2574 Da, and that chromatographed similar to ramoplanin A2. This single compound was purified to homogeneity, generating yields of 1-3 mg/L (isolated, unoptimized yields). This compound was named chersinamycin and bioinformatics-guided structure elucidation and evaluation of its antimicrobial activity and relationship to ramoplanin and enduracidin was evaluated.

In silico characterization of the chersinamycin BGC: To help reconcile the observed mass of chersinamycin with the predicted structure, the M. chersina DSM 44151 BGC was first examined, which is composed of 32 genes encoding proteins for transport, transcriptional regulation, amino acid biosynthesis, peptide assembly, and peptide tailoring (FIG. 7, Table 3).

TABLE 3
Deduced functions of proteins within the defined BGC of
Micromonospora chersina DSM 44151.
Bounds of the BGC as determined by SSN are shaded.

| Orf | Protein Product | Length | Protein Name |
|---|---|---|---|
| 1 | WP_091305412.1 | 586 | coagulation factor 5/8 type domain-containing protein |
| 2 | WP_091305414.1 | 1025 | hypothetical protein |
| 3 | WP_091305416.1 | 288 | hypothetical protein |
| 4 | WP_091305419.1 | 203 | hypothetical protein |
| 5 | WP_091305421.1 | 278 | hypothetical protein |
| 6 | WP_091305424.1 | 233 | hypothetical protein |
| 7 | WP_091305427.1 | 108 | hypothetical protein |
| 8 | WP_091321299.1 | 143 | YbaB/EbfC family DNA-binding protein |
| 9 | WP_091321301.1 | 333 | LacI family transcriptional regulator |
| 10 | WP_091305429.1 | 190 | hypothetical protein |
| 11 | WP_091305431.1 | 281 | methyltransferase domain-containing protein |
| 12 | WP_091305433.1 | 183 | hypothetical protein |
| 13 | WP_091305435.1 | 691 | licheninase |
| 14 | WP_091305439.1 | 447 | glycosyl hydrolase |
| 15 | WP_091305441.1 | 545 | ABC transporter ATP-binding protein |
| 16 | WP_091305445.1 | 382 | acyl-CoA dehydrogenase |
| 17 | WP_091305449.1 | 513 | long-chain fatty acid-CoA ligase |
| 18 | WP_091305452.1 | 490 | hypothetical protein |
| 19 | WP_091305455.1 | 632 | glycosyl transferase family 2 |
| 20 | WP_091305458.1 | 370 | beta-mannanase |
| 21 | WP_091321303.1 | 371 | beta-mannanase |
| 22 | WP_091305461.1 | 386 | beta-mannanase |
| 23 | WP_091305463.1 | 168 | hypothetical protein |
| 24 | WP_091305466.1 | 441 | aminotransferase class V-fold PLP-dependent enzyme |
| 25 | WP_091305469.1 | 299 | alpha/beta hydrolase |
| 26 | WP_091305472.1 | 209 | TetR family transcriptional regulator |
| 27 | WP_091305475.1 | 906 | helix-turn-helix transcriptional regulator |
| 28 | WP_091305478.1 | 330 | hypothetical protein |
| 29 | WP_091321305.1 | 412 | PLP-dependent aminotransferase family protein |
| 30 | WP_091321307.1 | 260 | enoyl-CoA hydratase |
| 31 | WP_091321309.1 | 425 | enoyl-CoA hydratase/isomerase family |
| 32 | WP_091321311.1 | 205 | enoyl-CoA hydratase |
| 33 | WP_091305480.1 | 384 | type III polyketide synthase |
| 34 | WP_091305483.1 | 339 | 4-hydroxyphenylpyruvate dioxygenase |
| 35 | WP_091321312.1 | 388 | aminohydrolase family protein |
| 36 | WP_091321314.1 | 639 | ABC transporter ATP-binding protein |
| 37 | WP_091305485.1 | 266 | alpha/beta hydrolase |
| 38 | WP_091305488.1 | 529 | MBLfold metallo-hydrolase |
| 39 | WP_091305490.1 | 90 | acyl carrier protein |
| Chers A | WP_091305493.1 | 2133 | amino acid adenylation domain-containing protein |
| Chers B | WP_091305496.1 | 6998 | amino acid adenylation domain-containing protein |
| Chers C | WP_091305499.1 | 8746 | amino acid adenylation domain-containing protein |
| 43 | WP_091305502.1 | 231 | thioesterase |
| 44 | WP_091305505.1 | 286 | NAD(P)-dependent oxidoreductase |
| Chers D | WP_091321316.1 | 898 | amino acid adenylation domain-containing protein |
| 46 | WP_091305507.1 | 209 | class I SAM-dependent methyltransferase |
| 47 | WP_091305509.1 | 178 | hypothetical protein |
| 48 | WP_091305512.1 | 468 | DUF2029 domain-containing protein |
| 49 | WP_091305514.1 | 531 | FAD-dependent oxidoreductase |
| 50 | WP_091305517.1 | 218 | DNA-binding response regulator |
| 51 | WP_091321318.1 | 359 | two-component sensor histidine kinase |
| 52 | WP_091305519.1 | 184 | hypothetical protein |
| 53 | WP_091305522.1 | 301 | ABC transporter ATP-binding protein |
| 54 | WP_091305525.1 | 584 | hypothetical protein |
| 55 | WP_091321320.1 | 73 | MbtH family protein |
| 56 | WP_091305529.1 | 59 | hypothetical protein |
| 57 | WP_091305532.1 | 442 | cation/H(+) antiporter |
| 58 | WP_091321322.1 | 127 | chorismate mutase |
| 59 | WP_091321324.1 | 633 | hypothetical protein |
| 60 | WP_091305536.1 | 352 | alpha-hydroxy-acid oxidizing enzyme |
| 61 | WP_091305540.1 | 252 | class I SAM-dependent methyltransferase |
| 62 | WP_091321326.1 | 759 | FAD-binding protein |
| 63 | WP_091305543.1 | 106 | antibiotic biosynthesis monooxygenase |
| 64 | WP_091305545.1 | 408 | cytochrome P450 |
| 65 | WP_091305548.1 | 221 | TcmI family type II polyketide cyclase |
| 66 | WP_091305551.1 | 221 | DUF2238 domain-containing protein |
| 67 | WP_091305553.1 | 127 | DUF1622 domain-containing protein |
| 68 | WP_091321328.1 | 158 | Appr-1-p processing protein |
| 69 | WP_091321330.1 | 280 | 4,5-DOPA dioxygenase extradiol |
| 70 | WP_091305555.1 | 709 | copper-translocating P-type ATPase |
| 71 | WP_091305557.1 | 133 | helix-turn-helix domain-containing protein |
| 72 | WP_091305559.1 | 259 | molybdate ABC transporter substrate-binding protein |
| 73 | WP_091305561.1 | 266 | molybdate ABC transporter permease subunit |
| 74 | WP_091321332.1 | 348 | ABC transporter ATP-binding protein |
| 75 | WP_091305563.1 | 580 | sulfatase |
| 76 | WP_091305566.1 | 325 | dehydrogenase |
| 77 | WP_091305569.1 | 132 | 6-carboxytetrahydropterin synthase |
| 78 | WP_091305571.1 | 345 | glycosyl transferase |
| 79 | WP_091305574.1 | 270 | SAM-dependent methyltransferase |
| 80 | WP_091305576.1 | 325 | dolichol-P-glucose synthetase-like protein |
| 81 | WP_091305578.1 | 211 | GTP cyclohydrolase II |

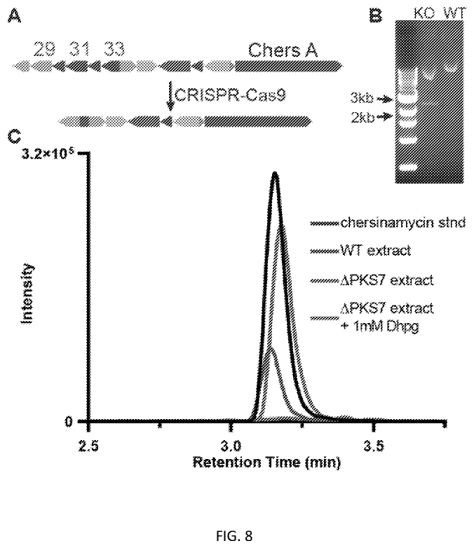

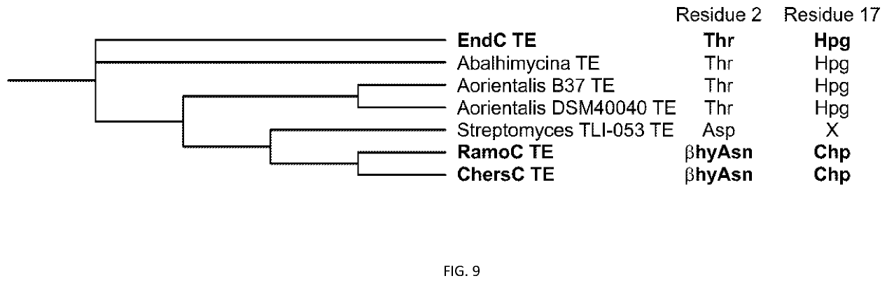

In addition to the four NRPSs A-D (Chers A-D) that are responsible for the production of a 17 residue linear peptide, the C-terminal thioesterase domain of Chers C suggests that the peptide is offloaded with concomitant cyclization (FIG. 8A, FIG. 9). While beta hydroxylation of the second amino acid, predicted as L-Asn, is difficult to predict based on adenylation domain sequence alone, a putative hydroxylase enzyme (Chers 38) was found in the chersinamycin BGC with high sequence identity to the ramoplanin hydroxylase (Ramo 10). A homologous enzyme is also identified in the Streptomyces sp. TLI_053 cluster, predicted to activate an aspartic acid at residue 2, but is absent in the additional four clusters which are each predicted to activate threonine at the second position (Table S2). Additionally, high percent identity between thioesterase sequences from the chersinamycin and ramoplanin clusters (FIG. 9) suggested the site of macrolactonization to be the same.

Turning to the surrounding chersinamycin biosynthetic machinery, the presence of genes for Hpg biosynthesis (Chers 29, 34, and 59) supports the large number of predicted Hpg residues in the peptide sequence (FIG. 7A, Table 2). At residues 4 and 10, the adenylation domain sequence confers specificity for a hydrophilic residue as predicted by NRPSPredictor2 (Table 2). The specificity sequences are nearly identical to those of ramoplanin and enduracidin at these positions, which contain Orn4, Orn10 and Orn4, End10, respectively. A lack of putative End biosynthesis proteins within the chersinamycin cluster led to the prediction of Orn4, Orn10 for chersinamycin.

Putative polyketide synthase-like (PKS-like) biosynthetic proteins Chers 29-33 with similarity to chalcone synthase and stilbene synthase suggested that chersinamycin may contain the amino acid dihydroxyphenylglycine (Dpg).⁶⁸ This amino acid is found within glycopeptides like vancomycin but absent in both ramoplanin and enduracidin. Though this residue was not directly predicted by NRPSPredictor2 or PKS/NRPS Analysis Web Site, an aromatic residue was predicted by NRPSPredictor 2 at Chers C-m4 (residue 13). Therefore, it was predicted that Dpg might be incorporated at residue 13, and that the Chers C may contain a novel Dpg-activating adenylation domain sequence.

N-acylation is essential to the antimicrobial activity of ramoplanin family antibiotics. In addition to the C^{III} domain of Chers A, a predicted FAAL (Chers 54) and ACP (Chers 39) are present within the cluster for fatty acid activation and transfer to the first NRPS-bound residue. Notably absent, however, was the prediction of putative ACADs (FIG. 7C, Table 4). While an oxidoreductase is present (Chers 22), a lack of these dehydrogenases in the chersinamycin cluster suggests either a different biosynthetic source for an unsaturated lipid, or the incorporation of a saturated lipid.

TABLE 4
Comparison of the ramoplanin-family gene clusters in seven bacterial strains.

| | | | | | A. | | |
|---|---|---|---|---|---|---|---|
| | | | | A. | orientalis | | |
| | | | M. | orientalis | DSM | A. | Streptomyces |
| | Enduracidin | Ramoplanin | chersina | B-37 | 40040 | bahlimycina | sp. TLI-053 |
| Acetyl-CoA | Orf 11 | | | | | Orf 12 43%^{a} | |
| acetyltransferase | | | | | | | |
| (thiolase) | | | | | | | |
| Transcriptional | Orf 12 | | | | | | |
| regulator | | | | | | | |
| β-Mannosidase | Orf 13 | | | | | | |
| Probable sugar | Orf 14 | | | | | | |
| transport system | | | | | | | |
| lipoprotein | | | | | | | |
| Sugar transport | Orf 15 | | | | | | |
| system permease | | | | | | | |
| protein | | | | | | | |
| Sugar transport | Orf 16 | | | | | | |
| system permease | | | | | | | |
| protein | | | | | | | |
| Ribonuclease D | Orf 17 | | | | | | |
| Two-component | Orf 18 | | | | | | |
| response regulator | | | | | | | |
| Unknown | Orf 19 | | | | | | |
| Uroporphyrinogen | Orf 20 | | | | | | |
| decarboxylase | | | | | | | |
| PAS protein | Orf 21 | | | | | | |
| phosphatase 2C-like | | | | | | | |
| Str-like regulatory | Orf 22 43%^{b} | Orf 5 43%^{a} | Orf 28 44%^{a} | Orf 29 54%^{a} | Orf 31 43%^{a} | Orf 29 53%^{a} | Orf 36 55%^{a} |
| protein | | | 72%^{b} | 45%^{b}, | 47%^{b}, | 46%^{b}, | 41%^{b} |
| | | | | Orf 30 44%^{a} | Orf 32 54%^{a} | Orf 30 45%^{a} | |
| | | | | 46%^{b} | 46%^{b} | 47%^{b} | |
| Prephenate | Orf 23 51%^{b} | Orf 4 51%^{a} | | | | | Orf 37 48%^{a} |
| dehydrogenase | | | | | | | 52%^{b}, |
| | | | | | | | Orf 77 57%^{a} |
| | | | | | | | 55%^{b} |
| Transcriptional | Orf 24 50%^{b} | Orf 5 49%^{a} | Orf 28 47%^{a} | Orf 29 49%^{a} | Orf 31 70%^{a} | Orf 29 50%^{a} | Orf 36 46%^{a} |
| regulator | | | 72%^{b} | 45%^{b}, | 47%^{b}, | 46%^{b}, | 41%^{b} |
| | | | | Orf 30 71%^{a} | Orf 32 49%^{a} | Orf 30 74%^{a} | |
| | | | | 46%^{b} | 46%^{b} | 47%^{a} | |
| 4- | Orf 25 48%^{b} | Orf 30 48%^{a} | Orf 34 41%^{a} | Orf 31 79%^{a} | Orf 30 80%^{a} | Orf 31 78% | Orf 54 42% |
| Hydroxyphenylpyruvate | | | 41%^{b} | 49%^{b} | 49%^{b} | 48%^{b} | 41%^{b} |
| dioxygenase (HmaS | | | | | | | |
| homologue) | | | | | | | |
| Unknown (MppR | Orf 26 | | | | | | |
| homologue) | | | | | | | |
| PLP-dependent | Orf 27 | | | | | | |
| aminotransferase | | | | | | | |
| (MppQ homologue) | | | | | | | |
| PLP-dependent | Orf 28 | | | | | | |
| aminotransferase | | | | | | | |
| (MppP homologue) | | | | | | | |
| Aminotransferase | Orf 29 | Orf 6 68%^{a}, | Orf 60 59%^{a}, | Orf 32 78%^{a} | Orf 29 78%^{a} | Orf 32 79%^{a} | Orf 53 67%^{a} |
| | | Orf 7 70%^{a} | Orf 29 70%^{a} | | | | |
| FAD-dependent | Orf 30 64%^{b} | Orf 20 64%^{a} | Orf 49 63%^{a} | Orf 34 83%^{a} | Orf 27 83%^{a} | Orf 34 84%^{a} | |
| oxidoreductase | | | 83%^{b} | 64%^{b} | 64%^{b} | 64%^{b} | |
| (halogenase) | | | | | | | |
| Transmembrane | Orf 31 | Orf 1 50%^{a}, | | Orf 35 71%^{a} | Orf 26 72%^{a} | Orf 35 73%^{a} | Orf 57 43%^{a} |
| transport protein | | Orf 3 56^{a} | | | | | |
| ABC transporter ATP- | Orf 32 | Orf 23 56%^{a}, | Orf 53 73%^{a} | Orf 36 78%^{a} | Orf 25 78%^{a} | Orf 36 81%^{a} | Orf 58 64%^{a} |
| binding protein | | Orf 2 71%^{a} | | | | | |
| ABC transporter | Orf 33 73%^{b} | Orf 8 73%^{a} | Orf 36 78%^{a} | Orf 37 78%^{a} | Orf 24 78%^{a} | Orf 37 79%^{a} | Orf 56 62%^{a} |
| | | | 77%^{b} | 74%^{b} | 74%^{b} | 75%^{b} | 63%^{b} |
| Alpha/beta fold | Orf 34 77%^{b} | Orf 9 77%^{a} | Orf 37 75%^{a} | Orf 38 71%^{a} | Orf 23 69%^{a} | Orf 38 76%^{a} | Orf 55 62%^{a} |
| hydrolase | | | 78%^{b} | 73%^{b} | 72%^{b} | 77%^{b} | 63%^{b} |
| MBL fold metallo- | | Orf 10 | Orf 38 82%^{b} | | | | Orf 48 72%^{b} |
| hydrolase | | | | | | | |
| Acyl carrier protein | Orf 35 69%^{b} | Orf 11 69%^{a} | Orf 39 63%^{a} | Orf 39 75%^{a} | Orf 22 76%^{a} | Orf 39 78%^{a} | Orf 43 54%^{a} |
| | | | 58%^{b} | 67%^{b} | 66%^{b} | 71%^{b} | 61%^{b} |
| NRPS A | End A 55%b | Ramo A 55%a | Orf 40 47%a | Orf 40 67%a | Orf 21 66%a | Orf 40 66%a | Orf 42 44%a |
| | | | 61%b | 54%b | 53%b | 55%b | 48%b |
| NRPS B | End B 62%b | Ramo B 62%a | Orf 41 68%a | Orf 41 70%a | Orf 20 70%a | Orf 41a 72%a | Orf 41 62%a |
| | | | 67%b | 61%b | 61%b | 66%b, | 60%b |
| | | | | | | Orf 41b 64%a | |
| | | | | | | 64%b | |
| NRPS C | End C 61%b | Ramo C 61%a | Orf 42 64%a | Orf 42 71%a | Orf 19 71%a | Orf 42 72%a | Orf 40 62%a |
| | | | 65%b | 61%b | 61%b | 61%b | 60%b |
| Thioesterase | EndC 66%^{b} | Orf 15 66%^{a} | Orf 43 70%^{a} | Orf 43) 79%^{a} | Orf 18 79%^{a} | Orf 43 83%^{a} | Orf 64 55%^{a} |
| | | | 70%^{b} | 64%^{b} | 65%^{b} | 64%^{b} | 53%^{b} |
| NAD(P)-dependent | Orf 39 80%^{b} | Orf 16 80%^{a} | Orf 44 81%^{a} | Orf 44 85%^{a} | Orf 17 85%^{a} | Orf 44 86%^{a} | Orf 63 69%^{a} |
| oxidoreductase | | | 84%^{b} | 78%^{b} | 79%^{b} | 78%^{b} | 71%^{b} |
| NRPS D | End D 57%b | Ramo D 57%a | Orf 45 63%a | Orf 45 67%a | Orf 16 67%a | Orf 45 69%a | Orf 62 46%a |
| | | | 63%a | 58%b | 57%b | 59%b | 46%b |
| Hypothetical protein | | Orf 18 | Orf 47 48%^{b} | | | | |
| GA0070603_0076 | | | | | | | |
| DUF2029 domain- | | Orf 19 | Orf 48 68%^{b} | | | | |
| containing protein | | | | | | | |
| DNA-binding | Orf 41 71%^{b} | Orf 21 71%^{a} | Orf 50 76%^{a} | Orf 46 74%^{a} | Orf 15 75%^{a} | Orf 46 77%^{a} | Orf 61 70%^{a} |
| response regulator | | | 82%^{b} | 70%^{b} | 71%^{b} | 73%^{b} | 70%^{b} |
| Sensor histidine | Orf 42 57%^{b} | Orf 22 57%^{a} | Orf 51 63%^{a} | Orf 47 72%^{a} | Orf 14 72%^{a} | Orf 47 74%^{a} | |
| kinase | | | 61%^{b} | 55%^{b} | 55%^{b} | 56%^{b} | |
| Two-component | Orf 43 | | | Orf 48 56%^{a} | Orf 13 56%^{a} | Orf 48 55%^{a} | |
| sensor histidine | | | | | | | |
| kinase | | | | | | | |
| Acyl-coA | Orf 44 67%^{b} | Orf 24 67%^{a} | | Orf 50 79%^{a} | Orf 11 78%^{a} | Orf 49 78%^{a} | Orf 44 69%^{a} |
| dehydrogenase | | | | 57%^{b} | 66%^{b} | 65%^{b} | 67%^{b} |
| Acyl-CoA ligase | Orf 45 54%^{b} | Orf 26 54%^{a} | Orf 54 62%^{a} | Orf 52 69%^{a} | Orf 9 69%^{a} | Orf 51 69%^{a} | Orf 46 51%^{a} |
| (FAAL) | | | 63%^{b} | 59%^{b} | 59%^{b} | 59%^{b} | 54%^{b} |
| Acyl-CoA | Orf 45 64%^{b} | Orf 25 64%^{a} | | Orf 51 74%^{a} | Orf 10 74%^{a} | Orf 50 78%^{a} | Orf 45 69%^{a} |
| dehydrogenase | | | | 65%^{b} | 65%^{b} | 65%^{b} | 64%^{b} |
| MbtH-like protein | Orf 46 89%^{b} | Orf 27 89%^{a} | Orf 55 91%^{a} | Orf 53 90%^{a} | Orf 8 90%^{a} | Orf 52 91%^{a} | Orf 47) 82%^{a} |
| | | | 93%^{b} | 87%^{b} | 87%^{b} | 88%^{b} | 82%^{b} |
| Chorismate mutase | | Orf 28 | Orf 58 65%^{b} | | | | |
| Glycosyltransferase | | Orf 29 | Orf 59 59%^{b} | Orf 49 55%^{b} | Orf 12 64%^{b} | | |
| Integral membrane | Orf 47 | | | | | | |
| protein | | | | | | | |
| Integral membrane | Orf 48 | | | | | | |
| protein | | | | | | | |
| Putative membrane | | Orf 31 | Orf 57 34%^{b} | | | | |
| antiporter | | | | | | | |

Percent identities are shown for proteins encoded by each Orf compared to the
^{a}enduracidin BGC and
^{b}ramoplanin BGCs.
NRPSs are bolded.

Additional ORFs within the BGC appear to encode halogenase and glycosyltransferase tailoring enzymes. Chers 49 is homologous to the characterized halogenases found within the ramoplanin and enduracidin BGCs (Ramo 20 and End 30). Genetic knockout and complementation of Ramo 20 and End 30 within their respective clusters demonstrated that these enzymes are responsible for the monochlorination of Hpg17 in ramoplanin and dichlorination of Hpg13 in enduracidin. Identical adenylation domain specificity sequences at these sites and altered halogenation patterns resulting from genetic replacement of End 30 with Ramo 20 in S. fungicidicus suggested that site specificity of halogenation is controlled by the local structural environment of the full peptide, rather than loading of a halogenated residue onto the NRPS. Confidently predicting the location of possible halogenated residues for chersinamycin was therefore not possible, but the high sequence similarity of Chers 49 to Ramo 20 and End 30 led to the belief in chlorination of an aromatic residue. Finally, the chersinamycin BGC contains a putative mannosyltransferase, Chers 59. The ramoplanin mannosyltransferase, Ramo 29, has been implicated through genetic knockout and complementation to instill two D-mannose sugars onto the phenolic oxygen of Hpg and therefore mono or diglycosylation was predicted for chersinamycin as well.

Chersinamycin isolation and structure elucidation: Numerous analytical methods were employed for the full structure elucidation of chersinamycin. HR-LC/MS revealed a [M+2H]²⁺ molecular ion of 1287.0511, suggesting a molecular formula of C₁₁₉H₁₅₈ClN₂₁O₄₁. The peptide macrocycle was determined to be highly base labile, with exposure to 1% triethylamine in water resulting in hydrolysis ([M+2H]²⁺ molecular ion 1296.044). This suggested a lactone macrocycle as opposed to a lactam which would remain intact under such weakly basic conditions, supporting the prediction that ring closure occurs at a side chain hydroxyl. The ¹H-NMR of the cyclic peptide showed a large number of exchangeable amide protons (δH 7.0-10.0) and signals within the a-proton region (δH 3.5-7.0), as well as many doublets in the aromatic region consistent with numerous Hpg residues (δH 6.0-7.5). Analysis of 2D NMR data allowed the assignment of the 17 amino acid residues (Table 5).

TABLE 5
NMR spectroscopic data of chersinamycin

| Residue | NH | α | β | other |
|---|---|---|---|---|
| Asn1 | 7.91 | 4.29 | 2.05, 1.74 | — |
| hyAsn2 | 8.26 | 5.27 | 5.55 | |
| Hpg3 | 9.58 | 5.98 | — | b/f 7.34; c/e 6.88 |
| Orn4 | 9.05 | 4.10 | 1.22, 1.08 | γ 1.37, δ 2.68, 2.47 |
| Thr5 | 7.43 | 4.17 | 3.89 | γ 0.94 |
| Hpg6 | 8.80 | 6.63 | — | b/f 6.52; c/e 6.19 |
| Hpg7 | 8.80 | 5.27 | — | b/f 6.52; c/e 6.30 |
| Thr8 | 8.13 | 3.56 | 3.76 | γ 0.59 |
| Phe9 | 7.47 | 4.01 | 2.05, 1.75 | b/f 6.80; c/e 7.09; d 7.04 |
| Orn10 | 7.60 | 4.81 | 1.91, 1.83 | γ 1.54; δ 2.88, 2.82 |
| Hpg11 | 9.10 | 6.80 | — | b/f 7.18; c/e 6.75 |
| Thr12 | 8.93 | | 3.79 | γ 0.80 |
| Dpg13 | 8.57 | 5.79 | — | b/f 6.09; d 6.04 |
| Gly14 | 7.76 | 3.60, 2.94 | — | — |
| Val15 | 8.33 | 3.66 | 1.69 | γ 0.72 |
| Ala16 | 9.26 | 4.16 | 1.23 | — |
| Chp17 | 7.65 | 4.76 | — | b 6.20; e 6.67; f 6.35 |

| | |
|---|---|
| lipid | HC^{α} 1.97, HC^{β} 1.30, HC^{γ} 1.04, HC^{δ} 0.95, HC^{ε} 1.04, HC^{ζ} 0.95, HC^{η} 1.30, CH₃ 0.65 |

COSY and TOCSY correlations were used to assign full aliphatic residues, confirming the incorporation of valine, alanine, glycine, threonines and ornithines into the peptide. COSY correlations between aromatic resonances in conjunction with NOEs between these resonances and their amide and alpha protons allowed the assignment of full aromatic residues. Two diagnostic singlets at δH 6.04 and OH 6.09 suggested a Dpg residue, supporting predictions based on the Dpg biosynthetic proteins within the gene cluster. Correlations observed between several resonances in the region between OH 3.0-5.0 are consistent with the presence of sugar moieties which were hypothesized to be incorporated by Chers 59. Though exact resonances could not be assigned due to spectral overlap, resonances were identical to those observed in ramoplanin, which coupled with the presence of a putative mannosyltransferase within the BGC, suggests D-mannoses are incorporated.

Unlike the diagnostic spectra for the Z,E unsaturated lipids of ramoplanin and enduracidin, the 1H-NMR of chersinamycin showed a lack of vinylic protons, and 2D spectra lacked correlations spanning the aliphatic-to-olefinic region, supporting the hypothesis of a saturated lipid based on the lack of ACADs in the gene cluster. To confirm saturation, chersinamycin was additionally subjected to catalytic hydrogenation. While hydrogenation of ramoplanin reduces both olefins resulting in a mass increase of 4 Da, no change was observed for chersinamycin after 24 hours under hydrogenation conditions. The 1H NMR does display a strong doublet at δH 0.65 indicating a terminally branched lipid.

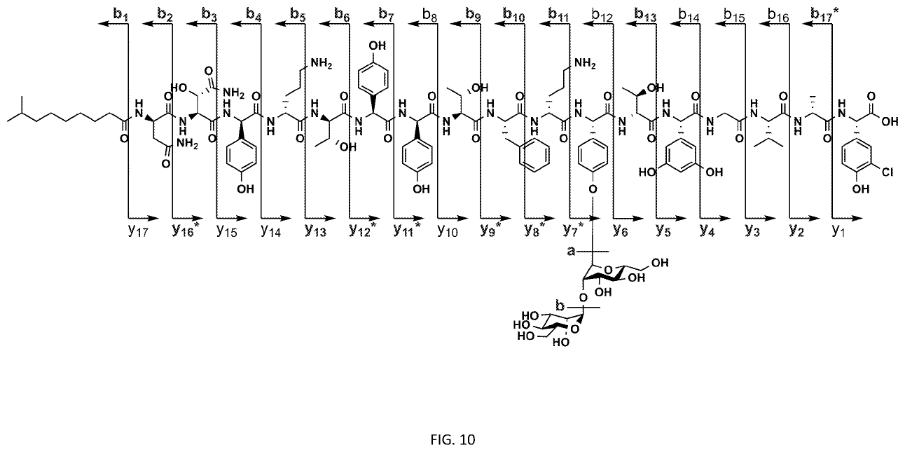

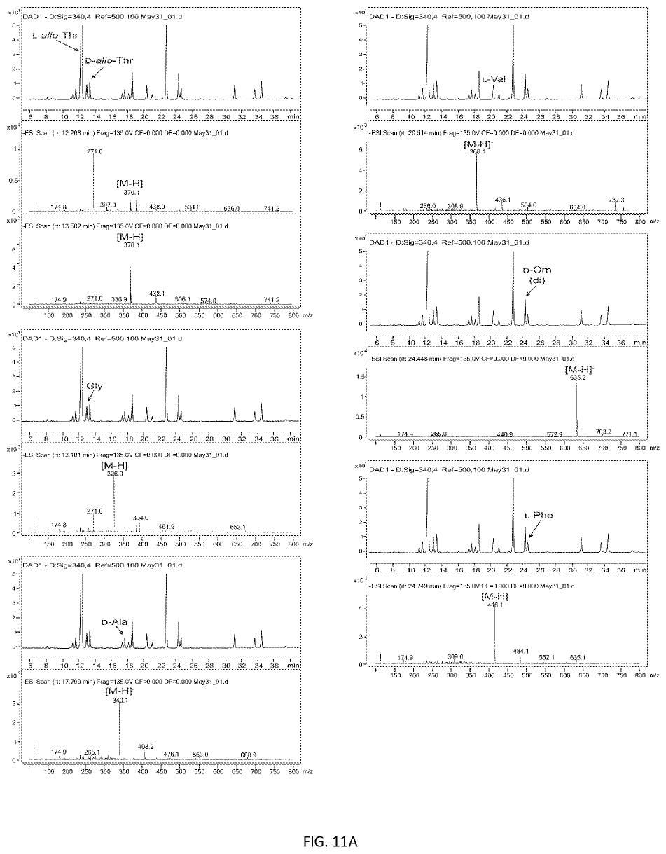

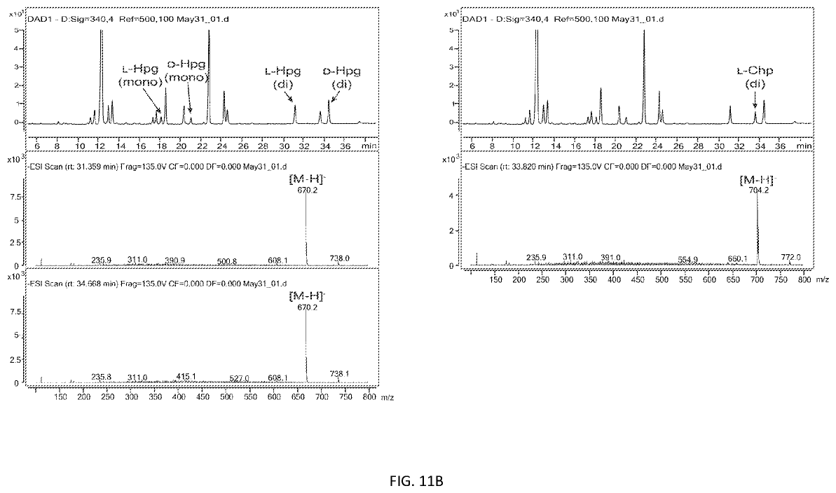

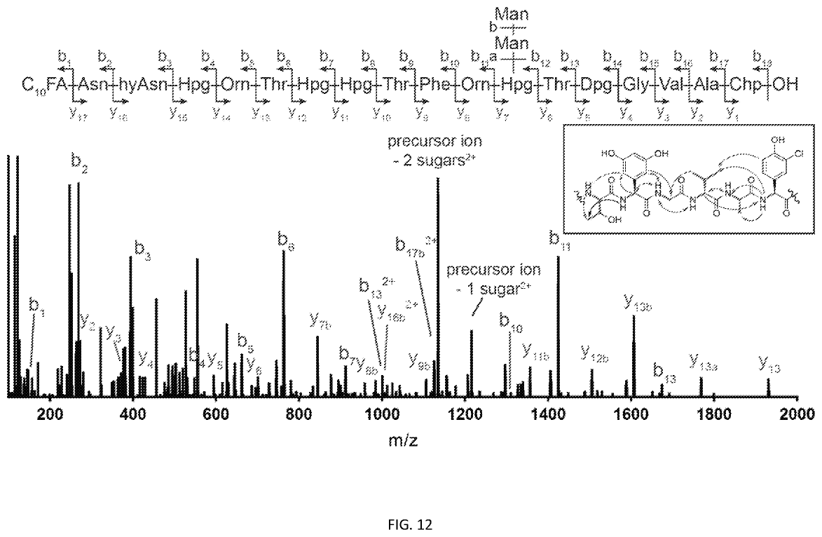

The peptide sequence hypothesized from in silico analysis of the chersinamycin NRPS domains was supported through analysis of the NOESY spectrum. NOEs between adjacent amide protons and between amide protons and adjacent alpha/beta protons allowed for connectivity to be determined. Strong NOE correlations between residues 2 and 17 supported macrolactonization between these residues as had been predicted through bioinformatics. To further validate connectivity, MS/MS was performed. Fragmentation focused on the molecular ion [M+2H]²⁺ (1287.05) resulted in two highly abundant doubly charged product ions of 1206.013 and 1124.986, each consistent with a loss of a mannose residue from the core peptide. Unfortunately, the high fragmentation energy required to fragment the peptide resulted in many ions that were not diagnostic, a common occurrence with cyclic and glycosylated peptides. MS/MS of acyclic chersinamycin focused on the molecular ion [M+2H]²⁺ (1296.04) resulted in a more simplified spectrum (FIG. 10, FIG. 11). Assignment of a number of b- and y-ions validated that hydrolysis occurred between residues 2 and 17, and confirmed the connectivity shown in FIG. 12.

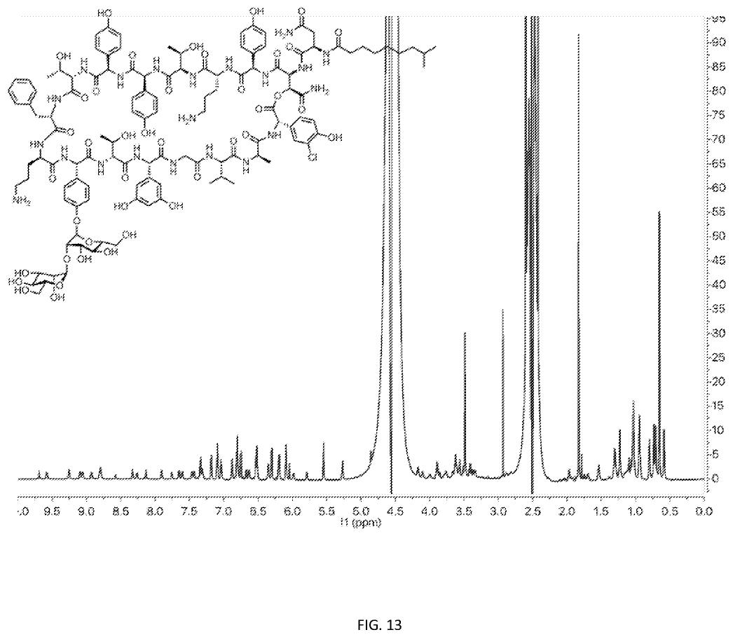

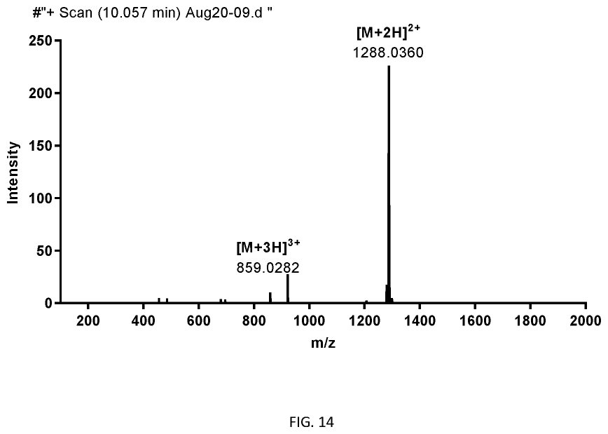

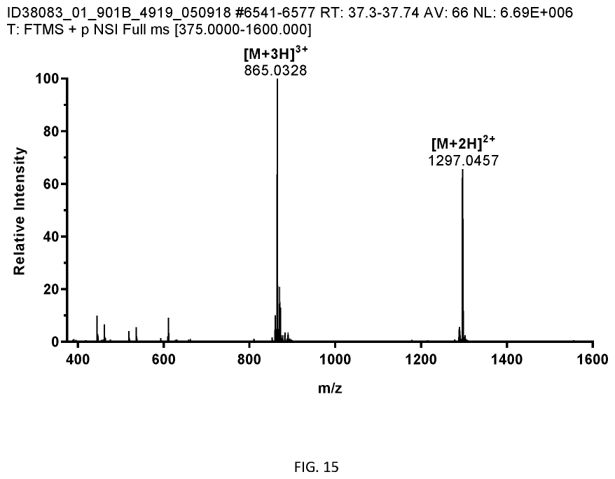

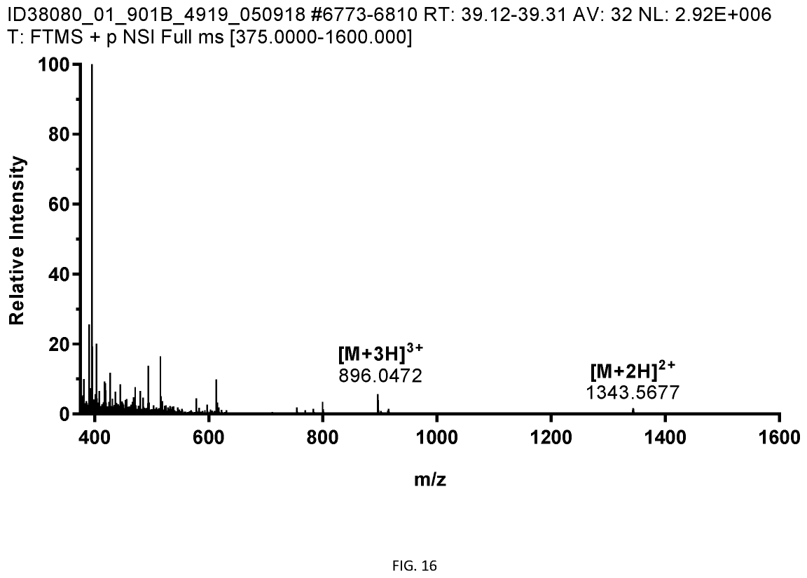

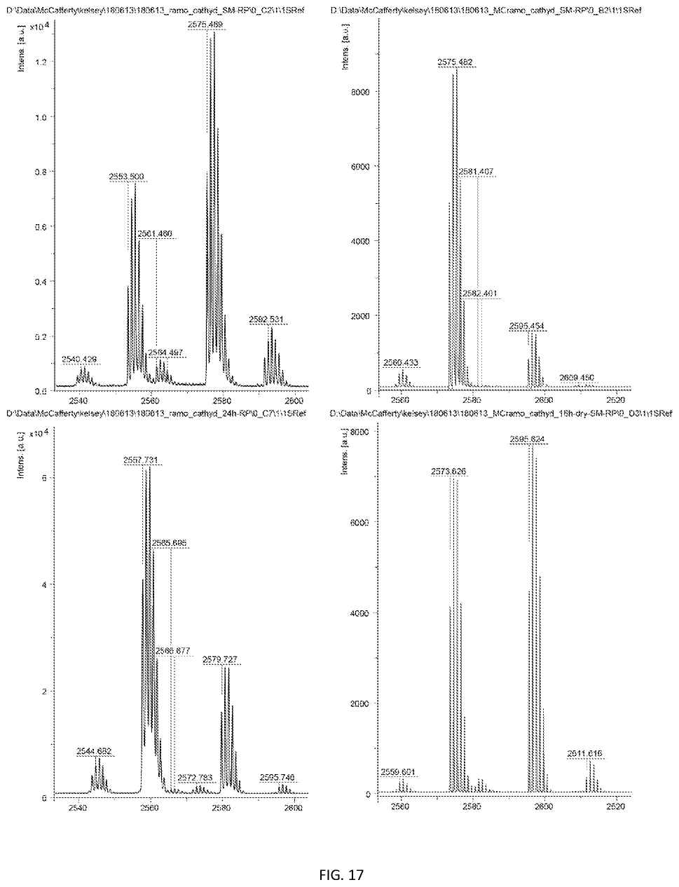

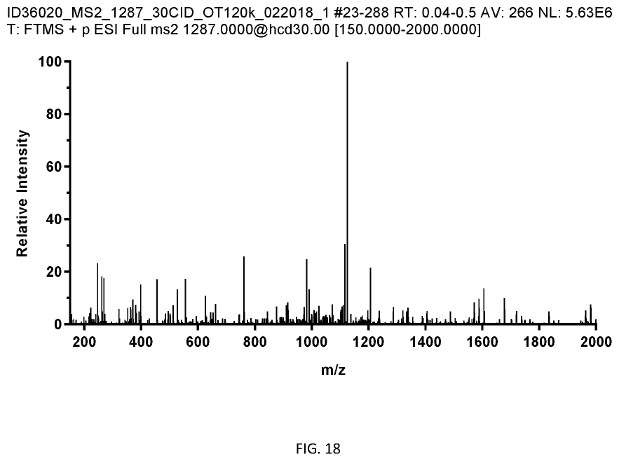

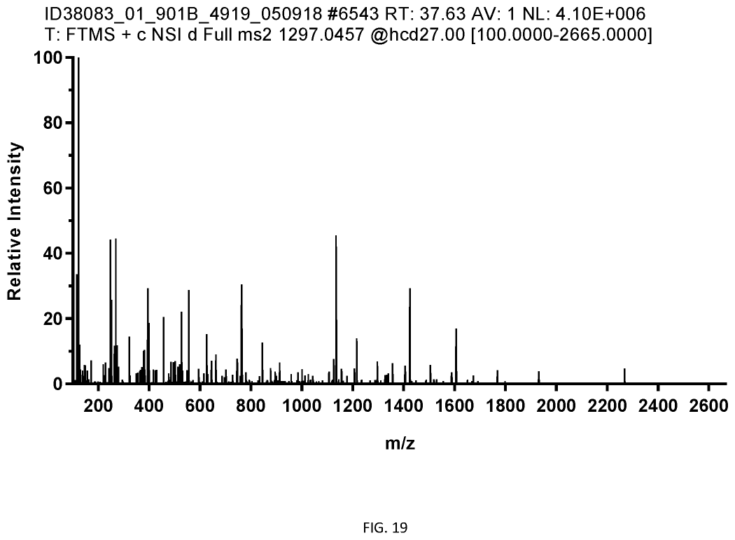

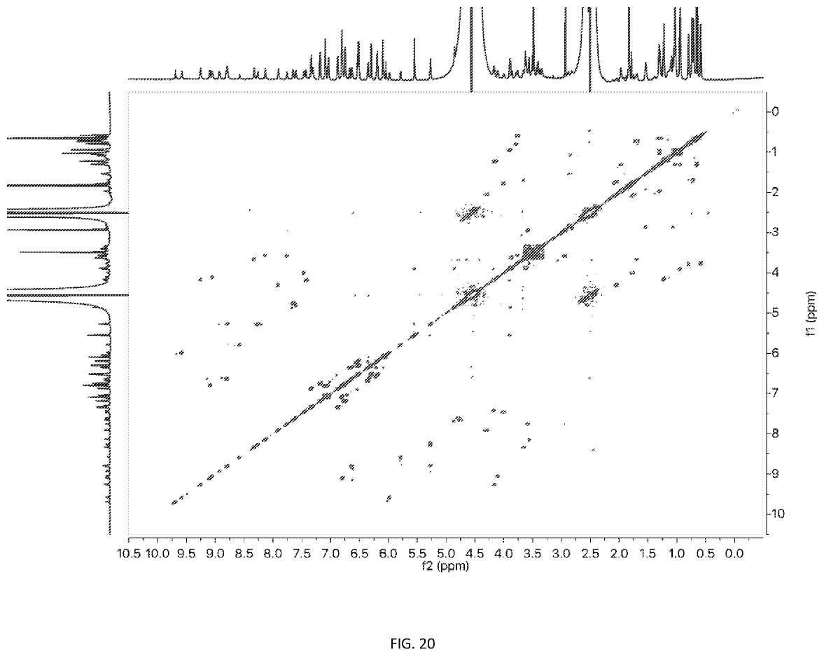

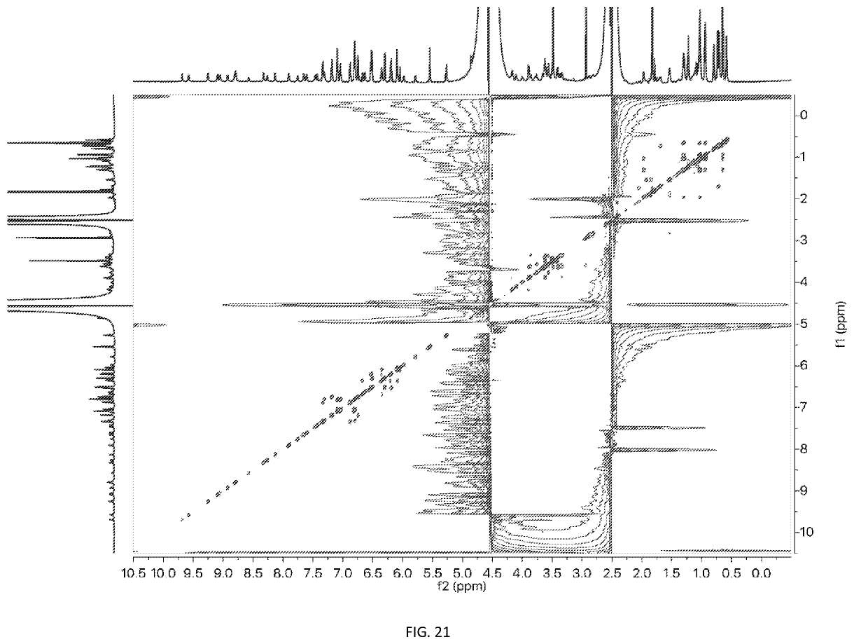

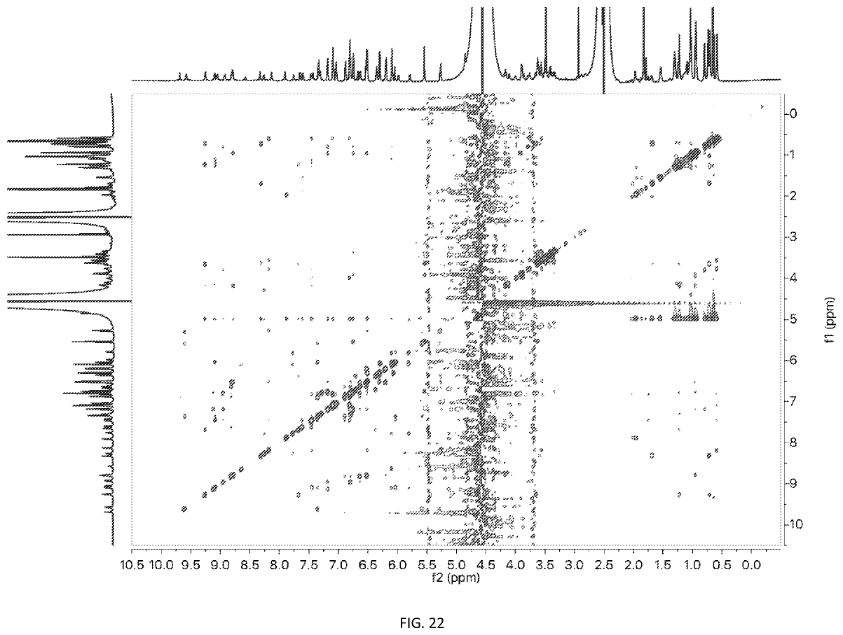

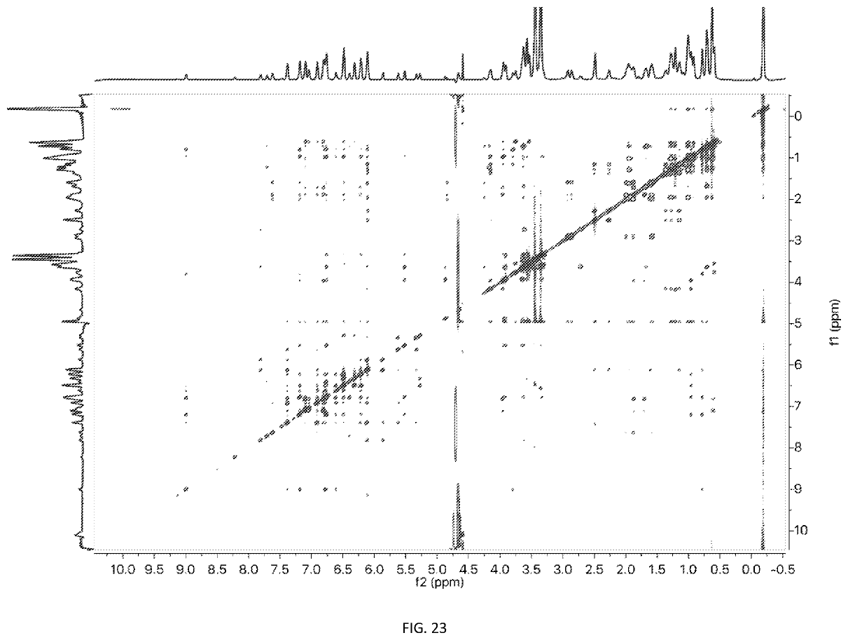

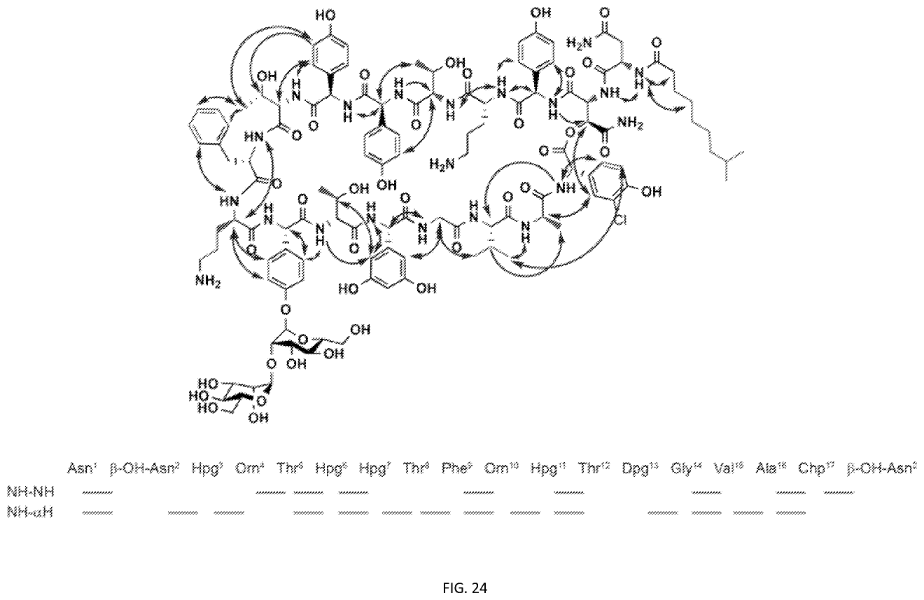

Advanced Marfey's analysis was employed to confirm the absolute configuration of each amino acid. Following complete hydrolysis and derivatization with Marfey's reagent (FDAA), the hydrolysate of chersinamycin was analyzed by LC-MS and peaks were compared to authentic standards of FDAA-amino acids (FIG. 13). It was determined that alanine and both ornithines are D-amino acids and valine, phenylalanine, and chlorohydroxyphenylglycine are L-amino acids. A 1:1 ratio of D-Hpg:L-Hpg was observed. This chromatography method was able to unambiguously distinguish DL-Thr from DL-allo-Thr, allowing for assignation of all threonines in chersinamycin as D-allo- and L-allo-Thr. The positions of D/L-amino acids in which both stereoisomers are present were assigned based on the analysis of the NRPS C/E domains. Unfortunately, asparagine and dihydroxyphenylglycine could not be identified in the FDAA-hydrolysate. As such, confirmation of absolute configuration of these residues was not possible, and assigned stereochemistry is based on the presence or absence of C/E domains.

Cumulatively, the bioinformatics analyses paired with analytical structure elucidation assigns the 2574 Da peptide from M. chersina as a 17-amino acid cyclic lipoglycodepsipeptide. The presence and location of D- and L-amino acids suggests chersinamycin's 3D structure to be very similar to ramoplanin and enduracidin. Unique from ramoplanin and enduracidin, chersinamycin exhibits a saturated N-acyl lipid and a noncanonical Dpg residue within the peptide sequence. The observation of glycosylation is an advantageous structural feature for solubility, stability and possible drug development. With the structure elucidated, the next goal was to unambiguously confirm the BGC and establish antimicrobial activity

Validation of the chersinamycin BGC using CRISPR-Cas9 gene editing: To confirm that the M. chersina BGC identified by genome mining was responsible for chersinamycin production, an LC-MS screen of the knockout strain M. chersina APKS7 was performed.⁶⁹ This mutant strain contains a 5.297 kilobase knockout of five genes encoding the putative biosynthesis enzymes for Dpg (Chers 29-33, FIG. 8A, 7B). Deletion of these biosynthetic genes resulted in the inability of M. chersina to produce chersinamycin. The knockout phenotype was rescued by the addition of 1 mM Dpg to the production medium (FIG. 8C). These studies establish the identity of the chersinamycin BGC and, importantly, demonstrated feasibility of CRISPR-mediated manipulation of this cluster.

Assessment of antimicrobial activity of chersinamycin: Chersinamycin was examined for its ability to inhibit bacterial growth by broth microdilution assays against Gram-positive strains B. subtilis ATCC 6051, S. aureus ATCC 25923, and E. faecalis ATCC 29212 and Gram-negative strain E. coli ATCC 25922. Chersinamycin was found to be ineffective against E. coli but have potent antimicrobial activity against the Gram-positive strains (Table 6).

TABLE 6
MICs of ramoplanin and chersinamycin

| | |
|---|---|
| Ramoplanin | Chersinamycin |

| | | | | |
|---|---|---|---|---|
| B. subtilis ATCC | <0.125 | μg mL⁻¹ | <0.125 | μg mL⁻¹ |
| 6051 | | | | |
| S. aureus ATCC | 0.5 | μg mL⁻¹ | 2 | μg mL⁻¹ |
| 25923 | | | | |
| E. faecalis ATCC | 0.5 | μg mL⁻¹ | 1 | μg mL⁻¹ |
| 29212 | | | | |
| E. coli ATCC | >64 | μg mL⁻¹ | >64 | μg mL⁻¹ |
| 25922 | | | | |

Due to its structural similarities to ramoplanin, it is expected that Chersinamycin will have activity against important clinically relevant pathogens such as C. difficile as well. As such, chersinamycin provides an additional potent ramoplanin family antibiotic for investigation into its antimicrobial potency and pharmacokinetic properties.

### Discussion/Conclusions:

The emergence of resistance to nearly all first line antibiotics has put enormous pressure on the development of new therapeutics. Ramoplanin is a potent antibiotic that is bactericidal against a number of clinically relevant Gram-positive pathogens, but poor bioavailability and stability highlight a need for development next generation analogs with better pharmacological properties. Described herein is a targeted genome mining strategy that is able to rapidly and reliably identify ramoplanin family gene clusters using established SAR. This has resulted in the discovery of five previously unidentified ramoplanin family BGCs in five additional bacterial strains. Of the strains identified, four have been previously cultured and extracted for other biologically active natural products, highlighting the importance of precise screening and extraction methods in identifying new natural products, and the significance of genome mining in natural product discovery. Bioinformatic analyses of putative proteins within the gene clusters allowed for structural predictions of the encoded natural products. These analyses predict 17-residue lipoglycodepsipeptides (from M. chersina and A. orientalis strains) and lipodepsipeptides (from A. balhimycina and Streptomyces sp. TLI_053) with high sequence similarity to ramoplanin and enduracidin, providing further support of the significance of certain structural features for this class of antibiotics. Bettering understanding of SAR through such analyses will aid in more insightful design of new antibiotics with improved biological properties.

To validate one of the five identified biosynthetic gene clusters involved in the production of a ramoplanin congener, the new antibiotic chersinamycin was isolated from fermentation of M. chersina. Its covalent structure was evaluated, and CRISPR-Cas9 gene editing approaches were used to validate that this gene cluster produces chersinamycin. Thorough bioinformatic analysis paired with classical structure determination approaches allowed for structure elucidation, thus expanding this important antibiotic class for the first time since the discovery of ramoplanin over three decades ago. Chersinamycin retains many of the structural features of ramoplanin, including the presence of two mannose sugars which have been demonstrated to contribute to ramoplanin's stability and improved solubility over its sister compound enduracidin. The peptide was determined to have a saturated N-acyl lipid, contrasting the lipid structures of the other two characterized compounds within this family and consistent with the lack of dehydrogenases within the identified gene cluster. Interestingly, the gene cluster retains the oxidoreductase (Chers 44) which has been hypothesized to play a role in lipid unsaturation. Therefore, further investigation is needed to understand the lipid biosynthetic pathway in this antibiotic class, greater understanding of which may aid in the development of biosynthetic analogs with new lipid architectures of decreased hemolytic activity.

Finally, the isolation of a ramoplanin family compound from a genetically tractable strain provides exciting opportunities for investigation of the biosynthetic pathway and development of biosynthetic analogs. A CRISPR-Cas9 strategy has been developed to produce a series of gene-inactivation mutants throughout the genome of M. chersina, a strategy that is difficult to achieve in many strains of natural product-producing organisms. Herein it is demonstrated that one such mutant strain, M. chersina APKS7, contains a knockout of the Dpg biosynthesis genes within the chersinamycin BGC that abolishes chersinamycin production. The ability to rescue production through supplementation of Dpg in the production medium demonstrates the feasibility of CRISPR-mediated manipulation of this biosynthetic pathway. This work therefore presents exciting opportunities for targeted gene inactivation to investigate enzymes within the chersinamycin biosynthetic pathway, as well as to produce biosynthetic analogs.

### Additional Tables

Additional tables relevant to the data described above are provided below.

TABLE 7
List of calculated and observed b- and y-
ions from MS/MS of acyclic chersinamycin

| | |
|---|---|
| calculated | observed |

| b ions | M + 1 | M + 2 | M + 1 | M + 2 |
|---|---|---|---|---|
| 1 | 155.144 | | 155.144 | |
| 2 | 269.187 | | 269.187 | |
| 3 | 399.224 | | 399.121 | |
| 4 | 548.272 | | 548.275 | |
| 5 | 662.351 | | 662.359 | |
| 6 | 763.400 | | 763.394 | |
| 7 | 912.447 | | 912.445 | |
| 8 | 1061.494 | 531.251 | | |
| 9 | 1162.542 | 581.774 | 1162.517 | |
| 10 | 1309.615 | 655.309 | 1310.609 | |
| 11 | 1423.690 | 712.384 | 1423.693 | |
| 12 | 1896.843 | 948.925 | | |
| 13 | 1997.891 | 999.950 | | 999.902 |
| 14 | 2162.933 | 1082.476 | | |
| 15 | 2219.955 | 1110.983 | | |
| 16 | 2319.023 | 1160.517 | | |
| 17 | 2390.060 | 1196.035 | | |
| 18 | 2573.069 | 1287.540 | | |
| 12a | 1734.790 | 867.899 | | |
| 13a | 1835.837 | 918.422 | | |
| 14a | 2000.887 | 1001.445 | | |
| 15a | 2057.902 | 1029.956 | | |
| 16a | 2156.967 | 1079.490 | | |
| 17a | 2228.007 | 1115.009 | | |
| a | 2428.019 | 1215.015 | | 1215.022 |
| 12b | 1572.737 | 786.872 | | |
| 13b | 1673.785 | 837.396 | 1673.785 | |
| 14b | 1838.827 | 919.917 | | |
| 15b | 1895.849 | 948.428 | | |
| 16b | 1994.917 | 998.464 | | |
| 17b | 2065.954 | 1033.982 | | 1033.981 |
| b | 2265.967 | 1133.988 | | 1134.026 |

| | |
|---|---|
| calculated | observed |

| y ions | M + 1 | M + 2 | M + 1 | M + 2 |
|---|---|---|---|---|
| 1 | 202.027 | | | |
| 2 | 273.064 | | 273.064 | |
| 3 | 372.132 | | 372.129 | |
| 4 | 429.154 | | 429.154 | |
| 5 | 594.196 | | 594.194 | |
| 6 | 695.244 | | 695.242 | |
| 7 | 1168.397 | | | |
| 8 | 1282.476 | 641.742 | | |
| 9 | 1429.545 | 715.276 | | |
| 10 | 1530.593 | 765.800 | | |
| 11 | 1679.640 | 840.322 | | |
| 12 | 1828.688 | 914.848 | | |
| 13 | 1929.736 | 965.371 | 1929.748 | |
| 14 | 2083.815 | 1022.913 | | |
| 15 | 2192.863 | 1097.437 | | |
| 16 | 2322.900 | 1162.456 | | |
| 17 | 2436.943 | 1219.477 | | |
| 7a | 1006.344 | | 1006.347 | |
| 8a | 1120.423 | 560.716 | | |
| 9a | 1267.492 | 633.746 | | |
| 10a | 1368.539 | 684.774 | | |
| 11a | 1517.587 | 759.297 | | |
| 12a | 1666.635 | 833.821 | | |
| 13a | 1767.683 | 884.345 | 1767.670 | |
| 14a | 1881.762 | 941.385 | | |
| 15a | 2030.810 | 1016.410 | | |
| 16a | 2160.848 | 1081.429 | | |
| 17a | 2274.891 | 1138.451 | | |
| 7b | 844.292 | | 844.295 | |
| 8b | 958.371 | 479.689 | 958.372 | |
| 9b | 1105.439 | 553.233 | 1105.434 | |
| 10b | 1205.479 | 603.243 | | |
| 11b | 1355.535 | 678.271 | 1355.530 | |
| 12b | 1504.582 | 752.795 | 1504.582 | |
| 13b | 1605.630 | 803.319 | 1605.639 | |
| 14b | 1719.709 | 860.358 | | |
| 15b | 1868.757 | 934.882 | | |
| 16b | 1998.795 | 1000.403 | | 1000.405 |
| 17b | 2112.838 | 1057.424 | | |

afragment with loss of one sugar;
bfragment with loss of two sugars

TABLE 8
Retention times for FDAA derivatives of amino
acid standards and chersinamycin hydrolysate

| | L-AA-FDAA | D-AA-FDAA | hydrolysate |
|---|---|---|---|
| Thr | 11.75 | 15.17 | |
| allo-Thr | 12.27 | 13.53 | 12.37, 13.42 |
| FDAA | 12.31 | — | 12.37 |
| Gly | 12.853 | — | 13.03 |
| Ala | 14.73 | 17.67 | 17.71 |
| Hpg (mono) | 18.01 | 20.56 | 18.19, 20.43 |
| Val | 20.39 | 24.17 | 20.43 |
| Orn (di) | 25.75 | 24.10 | 24.35 |
| Phe | 24.71 | 24.34 | 24.67 |
| Hpg (di) | 31.29 | 34.54 | 31.29, 34.59 |
| ClHpg (di) | 34.08 | — | 33.75 |
| Asn | 10.71 | 10.90 | |
| Dpg (mono) | 16.21 | 17.14 | |
| Dpg (di) | 29.71 | 31.47 | 5 |

TABLE 9
Deduced functions of proteins within the defined BGC of
Amycolatopsis orientalis B37.
Bounds of the BGC as determined by SSN are shaded.

| Orf | Protein Product | Length | Protein Name |
|---|---|---|---|
| 1 | WP_044850665.1 | 315 | hypothetical protein |
| 2 | WP_044850664.1 | 751 | Cu(2+)-exporting ATPase |
| 3 | WP_044850663.1 | 235 | metal ABC transporter ATP-binding protein |
| 4 | WP_044850763.1 | 283 | metal ABC transporter permease |
| 5 | WP_044850662.1 | 403 | lipoprotein |
| 6 | WP_044850661.1 | 299 | zinc ABC transporter substrate-binding protein |
| 7 | WP_044850660.1 | 388 | hypothetical protein |
| 8 | WP_044850659.1 | 136 | hypothetical protein |
| 9 | WP_065912849.1 | 326 | hypothetical protein |
| 10 | WP_044850657.1 | 245 | hypothetical protein |
| 11 | WP_044850656.1 | 683 | NACHT domain-containing protein |
| 12 | WP_044850655.1 | 386 | cytochrome P450 |
| 13 | WP_044850654.1 | 176 | MarR family transcriptional regulator |
| 14 | WP_083254979.1 | 68 | hypothetical protein |
| 15 | WP_044850653.1 | 239 | SGNH hydrolase |
| 16 | WP_083254980.1 | 350 | LacI family transcriptional regulator |
| 17 | WP_044850652.1 | 510 | sugar ABC transporter ATP-binding protein |
| 18 | WP_044850651.1 | 341 | ABC transporter permease |
| 19 | WP_044850650.1 | 338 | ABC transporter permease |
| 20 | WP_044850649.1 | 357 | rhamnose ABC transporter substrate-binding protein |
| 21 | WP_044850648.1 | 391 | L-rhamnose isomerase |
| 22 | WP_044850647.1 | 676 | bifunctional rhamnulose-1-phosphate aldolase/short- |
| | | | chain dehydrogenase |
| 23 | WP_044850761.1 | 484 | rhamnulokinase |
| 24 | WP_044850646.1 | 139 | PaaI family thioesterase |
| 25 | WP_044850645.1 | 402 | riboflavin synthase subunit alpha |
| 26 | WP_044850644.1 | 143 | nuclear transport factor 2 family protein |
| 27 | WP_083254981.1 | 184 | TetR family transcriptional regulator |
| 28 | WP_044850643.1 | 307 | alpha/beta hydrolase |
| 29 | WP_052674858.1 | 332 | transcriptional regulator |
| 30 | WP_083255282.1 | 357 | streptomycin biosynthesis protein |
| 31 | WP_044850641.1 | 287 | 4-hydroxyphenylpyruvate dioxygenase |
| 32 | WP_052674849.1 | 789 | Aminotransferase |
| 33 | WP_044850640.1 | 778 | penicillin acylase family protein |
| 34 | WP_044850639.1 | 500 | FAD-dependent oxidoreductase |
| 35 | WP_065912850.1 | 341 | transmembrane transport protein |
| 36 | WP_044850637.1 | 308 | ABC transporter ATP-binding protein |
| 37 | WP_083254982.1 | 650 | ABC transporter ATP-binding protein |
| 38 | WP_044850636.1 | 275 | alpha/beta hydrolase |
| 39 | WP_044850635.1 | 90 | acyl carrier protein |
| 40 | WP_052674848.1 | 2091 | non-ribosomal peptide synthetase |
| 41 | WP_065912851.1 | 7005 | non-ribosomal peptide synthetase |
| 42 | WP_065912852.1 | 8696 | non-ribosomal peptide synthetase |
| 43 | WP_044850632.1 | 236 | thioesterase |
| 44 | WP_044850631.1 | 274 | NAD(P)-dependent oxidoreductase |
| 45 | WP_083254983.1 | 861 | amino acid adenylation domain-containing protein |
| 46 | WP_044850630.1 | 221 | DNA-binding response regulator |
| 47 | WP_083254984.1 | 421 | sensor histidine kinase |
| 48 | WP_044850753.1 | 169 | hypothetical protein |
| 49 | WP_083254985.1 | 373 | hypothetical protein |
| 50 | WP_044850629.1 | 554 | acyl-CoA dehydrogenase |
| 51 | WP_065912853.1 | 576 | acyl-CoA dehydrogenase |
| 52 | WP_083254986.1 | 618 | hypothetical protein |
| 53 | WP_037306096.1 | 74 | MbtH family protein |
| 54 | WP_044850628.1 | 458 | 1,4-beta-xylanase |
| 55 | WP_052674845.1 | 138 | FHA domain-containing protein |
| 56 | WP_044850627.1 | 184 | hemerythrin domain-containing protein |
| 57 | WP_044850626.1 | 178 | hypothetical protein |
| 58 | WP_044850748.1 | 179 | N-acetyltransferase |
| 59 | WP_044850625.1 | 390 | pyridoxal phosphate-dependent aminotransferase |
| 60 | WP_052674844.1 | 371 | hypothetical protein |
| 61 | WP_083254987.1 | 470 | hypothetical protein |
| 62 | WP_083254988.1 | 338 | methyltransferase domain-containing protein |
| 63 | WP_044850623.1 | 421 | transcriptional regulator |
| 64 | WP_044850622.1 | 404 | hypothetical protein |
| 65 | WP_044850621.1 | 371 | radical SAM protein |
| 66 | WP_065912854.1 | 695 | hypothetical protein |
| 67 | WP_083254989.1 | 384 | KR domain-containing protein |
| 68 | WP_044850619.1 | 274 | ROK family protein |
| 69 | WP_044850744.1 | 398 | DegT/DnrJ/EryC1/StrS family aminotransferase |
| 70 | WP_065912855.1 | 344 | gfo/ldh/MocA family oxidoreductase |
| 71 | WP_065912856.1 | 288 | hypothetical protein |
| 72 | WP_044850617.1 | 208 | PIG-L family deacetylase |
| 73 | WP_083255283.1 | 146 | 3-dehydroquinate dehydratase |
| 74 | WP_044850615.1 | 239 | hypothetical protein |
| 75 | WP_044850614.1 | 510 | hypothetical protein |
| 76 | WP_044850613.1 | 85 | acyl carrier protein |
| 77 | WP_083254990.1 | 778 | hypothetical protein |
| 78 | WP_044850612.1 | 447 | hypothetical protein |
| 79 | WP_044850611.1 | 225 | hypothetical protein |
| 80 | WP_044850610.1 | 268 | sulfate adenylyltransferase subunit CysD |
| 81 | WP_052674838.1 | 412 | hypothetical protein |

TABLE 10
Deduced functions of proteins within the defined BGC of
Amycolatopsis orientalis DSM 40040.
Bounds of the BGC as determined by SSN are shaded.

| Orf | Protein product | Length | Protein name |
|---|---|---|---|
| 1 | WP_037306093.1 | 898 | hypothetical protein |
| 2 | WP_037306377.1 | 134 | hypothetical protein |
| 3 | WP_037306094.1 | 184 | hypothetical protein |
| 4 | WP_037306378.1 | 681 | SARP family transcriptional regulator |
| 5 | WP_051173832.1 | 1098 | hypothetical protein |
| 6 | WP_081736288.1 | 188 | FHA domain-containing protein |
| 7 | WP_037306095.1 | 458 | 1,4-beta-xylanase |
| 8 | WP_037306096.1 | 74 | MbtH family protein |
| 9 | WP_081736289.1 | 618 | hypothetical protein |
| 10 | WP_081736299.1 | 567 | acyl-CoA dehydrogenase |
| 11 | WP_051173836.1 | 554 | acyl-CoA dehydrogenase |
| 12 | WP_081736300.1 | 679 | hypothetical protein (mannosyltransferase) |
| 13 | WP_037306386.1 | 169 | hypothetical protein |
| 14 | WP_081736290.1 | 421 | sensor histidine kinase |
| 15 | WP_037306097.1 | 221 | DNA-binding response regulator |
| 16 | WP_081736301.1 | 859 | amino acid adenylation domain-containing protein |
| 17 | WP_037306099.1 | 274 | NAD(P)-dependent oxidoreductase |
| 18 | WP_037306100.1 | 236 | Thioesterase |
| 19 | WP_051173837.1 | 8720 | non-ribosomal peptide synthetase |
| 20 | WP_051173838.1 | 7005 | non-ribosomal peptide synthetase |
| 21 | WP_051173839.1 | 2091 | non-ribosomal peptide synthetase |
| 22 | WP_051173840.1 | 90 | polyketide synthase |
| 23 | WP_051173841.1 | 275 | alpha/beta hydrolase |
| 24 | WP_037306101.1 | 650 | ABC transporter ATP-binding protein |
| 25 | WP_051173842.1 | 308 | ABC transporter ATP-binding protein |
| 26 | WP_037306103.1 | 341 | Transporter |
| 27 | WP_037306105.1 | 500 | FAD-dependent oxidoreductase |
| 28 | WP_037306106.1 | 778 | penicillin acylase family protein |
| 29 | WP_037306109.1 | 795 | aminotransferase |
| 30 | WP_037306110.1 | 357 | 4-hydroxyphenylpyruvate dioxygenase |
| 31 | WP_081736302.1 | 287 | streptomycin biosynthesis protein |
| 32 | WP_037306397.1 | 332 | transcriptional regulator |
| 33 | WP_037306113.1 | 59 | hypothetical protein |
| 34 | WP_037306114.1 | 402 | 3,4-dihydroxy-2-butanone-4-phosphate synthase |
| 35 | WP_037306115.1 | 139 | PaaI family thioesterase |
| 36 | WP_037306116.1 | 397 | HAF repeat-containing protein |
| 37 | WP_081736291.1 | 623 | glycosyltransferase family 2 protein |
| 38 | WP_081736303.1 | 256 | class I SAM-dependent methyltransferase |
| 39 | WP_081736292.1 | 752 | hypothetical protein |
| 40 | WP_051173844.1 | 169 | hypothetical protein |
| 41 | WP_051173845.1 | 264 | sugar ABC transporter ATP-binding protein |
| 42 | WP_037306401.1 | 480 | rhamnulokinase |
| 43 | WP_037306120.1 | 676 | bifunctional rhamnulose-1 -phosphate aldolase/short-chain |
| | | | dehydrogenase |
| 44 | WP_037306121.1 | 391 | L-rhamnose isomerase |
| 45 | WP_051173846.1 | 357 | rhamnose ABC transporter substrate-binding protein |
| 46 | WP_037306123.1 | 338 | ABC transporter permease |
| 47 | WP_037306124.1 | 341 | ABC transporter permease |
| 48 | WP_037306125.1 | 510 | sugar ABC transporter ATP-binding protein |
| 49 | WP_081736293.1 | 350 | LacI family transcriptional regulator |
| 50 | WP_037306126.1 | 59 | hypothetical protein |
| 51 | WP_037306127.1 | 239 | SGNH hydrolase |
| 52 | WP_037306129.1 | 176 | MarR family transcriptional regulator |
| 53 | WP_037306131.1 | 386 | cytochrome P450 |
| 54 | WP_037306132.1 | 683 | NACHT domain-containing protein |
| 55 | WP_037306133.1 | 245 | hypothetical protein |
| 56 | WP_037306134.1 | 326 | hypothetical protein |
| 57 | WP_037306136.1 | 136 | hypothetical protein |
| 58 | WP_037306137.1 | 388 | hypothetical protein |
| 59 | WP_037306140.1 | 299 | zinc ABC transporter substrate-binding protein |
| 60 | WP_037306142.1 | 403 | lipoprotein |

TABLE 11
Deduced functions of proteins within the defined BGC of
Amycolatopsis balhimycina FH 1894.
Bounds of the BGC as determined by SSN are shaded.

| Orf | Protein product | Length | Protein name |
|---|---|---|---|
| 1 | WP_020647547.1 | 2277 | KR domain-containing protein |
| 2 | WP_084642199.1 | 1442 | beta-ketoacyl synthase |
| 3 | WP_020647549.1 | 105 | acyl carrier protein |
| 4 | WP_020647550.1 | 269 | alpha/beta hydrolase |
| 5 | WP_026469625.1 | 389 | glycosyl transferase |
| 6 | WP_020647552.1 | 155 | GNAT family N-acetyltransferase |
| 7 | WP_020647553.1 | 278 | SDR family NAD(P)-dependent oxidoreductase |
| 8 | WP_020647554.1 | 82 | hypothetical protein |
| 9 | WP_026469627.1 | 278 | histidinol-phosphatase |
| 10 | WP_020647556.1 | 316 | ATP-dependent DNA ligase |
| 11 | WP_020647557.1 | 131 | hypothetical protein |
| 12 | WP_020647558.1 | 398 | acetyl-CoA C-acyltransferase |
| 13 | WP_020647559.1 | 146 | transcriptional regulator |
| 14 | WP_020647560.1 | 1197 | glycosyl hydrolase |
| 15 | WP_026469628.1 | 257 | NmrA family transcriptional regulator |
| 16 | WP_020647562.1 | 122 | DoxX family protein |
| 17 | WP_020647563.1 | 63 | hypothetical protein |
| 18 | WP_043791531.1 | 261 | CoA ester lyase |
| 19 | WP_020647565.1 | 152 | GNAT family N-acetyltransferase |
| 20 | WP_020647566.1 | 391 | CoA transferase |
| 21 | WP_020647567.1 | 587 | hypothetical protein |
| 22 | WP_020647568.1 | 1737 | hypothetical protein |
| 23 | WP_020647569.1 | 1068 | hypothetical protein |
| 24 | WP_020647570.1 | 393 | hypothetical protein |
| 25 | WP_026469629.1 | 1518 | kelch repeat-containing protein |
| 26 | WP_020647572.1 | 424 | hypothetical protein |
| 27 | WP_020647573.1 | 86 | hypothetical protein |
| 28 | WP_020647574.1 | 946 | AfsR/SARP family transcriptional regulator |
| 29 | WP_020647576.1 | 340 | hypothetical protein |
| 30 | WP_084642014.1 | 298 | streptomycin biosynthesis protein |
| 31 | WP_020647578.1 | 349 | 4-hydroxyphenylpyruvate dioxygenase |
| 32 | WP_020647579.1 | 805 | hypothetical protein |
| 33 | WP_051183855.1 | 779 | penicillin acylase family protein |
| 34 | WP_026469635.1 | 500 | FAD-dependent oxidoreductase |
| 35 | WP_026469636.1 | 341 | hypothetical protein |
| 36 | WP_051183856.1 | 311 | ABC transporter ATP-binding protein |
| 37 | WP_084642200.1 | 613 | ABC transporter ATP-binding protein |
| 38 | WP_020647585.1 | 280 | hypothetical protein |
| 39 | WP_020647586.1 | 90 | acyl carrier protein |
| 40 | WP_084642015.1 | 2108 | amino acid adenylation domain-containing protein |
| 41 | — | — | — |
| 42 | WP_020638000.1 | 8715 | non-ribosomal peptide synthetase |
| 43 | WP_026468001.1 | 236 | thioesterase |
| 44 | WP_020638002.1 | 274 | NAD(P)-dependent oxidoreductase |
| 45 | WP_051183728.1 | 861 | amino acid adenylation domain-containing protein |
| 46 | WP_020638004.1 | 221 | DNA-binding response regulator |
| 47 | WP_020638005.1 | 420 | sensor histidine kinase |
| 48 | WP_020638006.1 | 170 | hypothetical protein |
| 49 | WP_020638007.1 | 566 | acyl-CoA dehydrogenase |
| 50 | WP_020638008.1 | 586 | acyl-CoA dehydrogenase |
| 51 | WP_084641135.1 | 620 | hypothetical protein |
| 52 | WP_020638010.1 | 74 | MbtH family protein |
| 53 | WP_026468003.1 | 219 | SAM-dependent methyltransferase |
| 54 | WP_020638012.1 | 311 | 1-phosphofructokinase |
| 55 | WP_020638013.1 | 369 | hypothetical protein |
| 56 | WP_020638014.1 | 102 | hypothetical protein |
| 57 | WP_020638015.1 | 151 | hypothetical protein |
| 58 | WP_020638016.1 | 352 | alcohol dehydrogenase |
| 59 | WP_020638017.1 | 555 | phosphoenolpyruvate-protein phosphotransferase |
| 60 | WP_026468004.1 | 94 | HPr family phosphocarrier protein |
| 61 | WP_026468005.1 | 253 | DeoR/GlpR transcriptional regulator |
| 62 | WP_020638021.1 | 212 | helix-turn-helix transcriptional regulator |
| 63 | WP_020638022.1 | 63 | hypothetical protein |
| 64 | WP_020638023.1 | 259 | thioesterase |
| 65 | WP_020638024.1 | 991 | amino acid adenylation domain-containing protein |
| 66 | WP_020638025.1 | 386 | hypothetical protein |
| 67 | WP_020638026.1 | 344 | GDP-mannose 4,6 dehydratase |
| 68 | WP_020638027.1 | 7658 | type I polyketide synthase |
| 69 | WP_051183729.1 | 779 | type I polyketide synthase |
| 70 | WP_084641138.1 | 210 | hypothetical protein |
| 71 | WP_020638032.1 | 2133 | type I polyketide synthase |
| 72 | WP_020638033.1 | 393 | cytochrome P450 |
| 73 | WP_020638034.1 | 62 | ferredoxin |
| 74 | WP_020638035.1 | 72 | hypothetical protein |
| 75 | WP_020638036.1 | 404 | cytochrome P450 |
| 76 | WP_020638037.1 | 351 | DegT/DnrJ/EryC1/StrS family aminotransferase |
| 77 | WP_020638038.1 | 459 | glycosyltransferase |
| 78 | WP_084642016.1 | 3830 | KR domain-containing protein |
| 79 | WP_084642017.1 | 258 | hypothetical protein |
| 80 | WP_020638041.1 | 1822 | type I polyketide synthase |

TABLE 12
Deduced functions of proteins within the defined BGC of
Streptomyces TLI-053.
Bounds of the BGC as determined by SSN are shaded.

| Orf | Protein product | Length | Protein name |
|---|---|---|---|
| 1 | WP_093859876.1 | 998 | DUF3893 domain-containing protein |
| 2 | WP_093859877.1 | 254 | phosphatidylserine synthase |
| 3 | WP_093859878.1 | 633 | DUF1998 domain-containing protein |
| 4 | WP_093859879.1 | 1271 | Helicase |
| 5 | WP_093859880.1 | 279 | hypothetical protein |
| 6 | WP_093859881.1 | 785 | hypothetical protein |
| 7 | WP_093859882.1 | 201 | hypothetical protein |
| 8 | WP_093859883.1 | 89 | hypothetical protein |
| 9 | WP_093859884.1 | 849 | DUF262 domain-containing protein |
| 10 | WP_093859885.1 | 1444 | hypothetical protein |
| 11 | WP_093864793.1 | 1072 | helicase |
| 12 | WP_093864794.1 | 406 | serine/threonine protein kinase |
| 13 | WP_093859886.1 | 312 | serine/threonine protein kinase |
| 14 | WP_093864795.1 | 718 | hypothetical protein |
| 15 | WP_093859887.1 | 140 | nuclear transport factor 2 family protein |
| 16 | WP_093859888.1 | 190 | PadR family transcriptional regulator |
| 17 | WP_093859889.1 | 363 | hypothetical protein |
| 18 | WP_093859890.1 | 909 | helix-turn-helix transcriptional regulator |
| 19 | WP_093864796.1 | 242 | DUF1275 domain-containing protein |
| 20 | WP_093859891.1 | 629 | amidohydrolase |
| 21 | WP_093859892.1 | 220 | hydrolase |
| 22 | WP_093859893.1 | 160 | DoxX family protein |
| 23 | WP_093859894.1 | 184 | DNA starvation/stationary phase protection protein |
| 24 | WP_093859895.1 | 278 | alpha/beta hydrolase |
| 25 | WP_093859896.1 | 192 | TetR/AcrR family transcriptional regulator |
| 26 | WP_093859897.1 | 292 | short-chain dehydrogenase |
| 27 | WP_093859898.1 | 492 | GMC family oxidoreductase |
| 28 | WP_093859899.1 | 162 | hypothetical protein |
| 29 | WP_093864797.1 | 460 | aspartate aminotransferase family protein |
| 30 | WP_093864798.1 | 480 | FAD-dependent oxidoreductase |
| 31 | WP_093859900.1 | 293 | LLM class flavin-dependent oxidoreductase |
| 32 | WP_093859901.1 | 109 | hypothetical protein |
| 33 | WP_093859902.1 | 213 | hypothetical protein |
| 34 | WP_093864799.1 | 188 | TetR family transcriptional regulator |
| 35 | WP_107452518.1 | 141 | hypothetical protein |
| 36 | WP_093864800.1 | 302 | transcriptional regulator |
| 37 | WP_093859903.1 | 365 | prephenate dehydrogenase/arogenate dehydrogenase |
| | | | family protein |
| 38 | WP_107452520.1 | 375 | hydroxyneurosporene methyltransferase |
| 39 | WP_093864801.1 | 266 | amidinotransferase |
| 40 | WP_093859905.1 | 8761 | non-ribosomal peptide synthetase |
| 41 | WP_093859906.1 | 7121 | amino acid adenylation domain-containing protein |
| 42 | WP_093859907.1 | 2139 | amino acid adenylation domain-containing protein |
| 43 | WP_093859908.1 | 90 | acyl carrier protein |
| 44 | WP_093859909.1 | 578 | acyl-CoA dehydrogenase |
| 45 | WP_093859910.1 | 581 | acyl-CoA dehydrogenase |
| 46 | WP_093859911.1 | 588 | hypothetical protein |
| 47 | WP_093859912.1 | 69 | MbtH family protein |
| 48 | WP_093859913.1 | 527 | MBL fold metallo-hydrolase |
| 49 | WP_093859914.1 | 268 | enoyl-CoA hydratase |
| 50 | WP_093859915.1 | 432 | enoyl-CoA hydratase/isomerase family protein |
| 51 | WP_093859916.1 | 219 | enoyl-CoA hydratase |
| 52 | WP_093859917.1 | 369 | type III polyketide synthase |
| 53 | WP_093859918.1 | 815 | aminotransferase |
| 54 | WP_093859919.1 | 337 | 4-hydroxyphenylpyruvate dioxygenase |
| 55 | WP_093859920.1 | 266 | alpha/beta hydrolase |
| 56 | WP_093859921.1 | 654 | ABC transporter ATP-binding protein |
| 57 | WP_093859922.1 | 330 | hypothetical protein |
| 58 | WP_093859923.1 | 300 | ABC transporter ATP-binding protein |
| 59 | WP_093859924.1 | 72 | hypothetical protein |
| 60 | WP_093859925.1 | 361 | hypothetical protein |
| 61 | WP_093859926.1 | 222 | DNA-binding response regulator |
| 62 | WP_093859927.1 | 988 | amino acid adenylation domain-containing protein |
| 63 | WP_093859928.1 | 274 | NAD(P)-dependent oxidoreductase |
| 64 | WP_093859929.1 | 236 | thioesterase |
| 65 | WP_093859931.1 | 108 | hypothetical protein |
| 66 | WP_063758125.1 | 123 | MULTISPECIES: hypothetical protein |
| 67 | WP_093859932.1 | 161 | hypothetical protein |
| 68 | WP_093859933.1 | 444 | MFS transporter |
| 69 | WP_093859934.1 | 264 | DUF1684 domain-containing protein |
| 70 | WP_093859935.1 | 286 | acyl-CoA thioesterase II |
| 71 | WP_093859936.1 | 257 | alpha-ketoglutarate-dependent dioxygenase AlkB |
| 72 | WP_093859937.1 | 271 | LysM peptidoglycan-binding domain-containing protein |
| 73 | WP_093859938.1 | 295 | hypothetical protein |
| 74 | WP_093859939.1 | 267 | hypothetical protein |
| 75 | WP_093859940.1 | 485 | ribosome biogenesis GTPase Der |
| 76 | WP_093859941.1 | 260 | (d)CMP kinase |
| 77 | WP_093859942.1 | 361 | prephenate dehydrogenase |
| 78 | WP_093859943.1 | 797 | DUF4139 domain-containing protein |
| 79 | WP_093859944.1 | 548 | DUF4139 domain-containing protein |
| 80 | WP_093859945.1 | 120 | DUF952 domain-containing protein |
| 81 | WP_107452522.1 | 374 | transcriptional regulator |

### Materials and Methods

General methods and materials. Bacterial cell culture media components were purchased from Affymetrix, Fisher Scientific, Millipore-Sigma, and BD Difco Laboratories. A sample of Pharmamedia was obtained from Archer Daniels Midland Company, and fish meal was purchased from Coyote Creek Organic Feed Mill and Farm. Ultra-high purity solvents were purchased from Millipore-Sigma and Fisher Scientific and used without further purification. All chemicals were purchased in their highest purity forms from Millipore-Sigma and used without further purification unless otherwise indicated. The 1D and 2D NMR spectra (COSY, TOCSY, NOESY) were collected on a Varian/Agilent DirectDrive2 spectrometer at 800 MHz. Preparative reverse-phase HPLC purifications were performed on a Waters Prep 150B system with a Phenomenex octadecyl silica (C18) column (250 mm×21 mm, 10 μm, 300 Å) or Vydac C18 column (250×10 mm, 5 μm, 300 Å). Analytical HPLC was performed on a Varian Prostar system with a Phenomenex C18 column (250×4.6 mm, 5 μm, 300 Å). Tandem MS/MS spectrometry was performed using a Fusion Lumos Orbitrap mass spectrometer. Matrix-assisted laser desorption time-of-flight mass spectrometry (MALDI-TOF) was performed using a Bruker Autoflex Speed LRF MALDI-TOF System. High-resolution mass spectra were collected on an Agilent 6224 LC/MS-TOF instrument.

Bioinformatics. The NCBI accession numbers for the ramoplanin and enduracidin biosynthetic gene loci are DD382878 and DQ403252, respectively. Using these sequences, seven ORFs encoding proteins or protein subdomains that correspond to functionally essential structural motifs conserved between both antibiotics that were determined by prior SAR studies served as probes for mining related genome sequences. NRPS A, NRPS B, NRPS C, NRPS D, the terminal thioesterase subdomain from NRPS C, the FAAL, and the ACP were used as initial queries for protein blast searches against the NCBI database. Sequences with >50% identity were collected and organisms that had four or more homologous proteins to the search queries were considered hits. Whole genome sequences for these organisms were obtained from NCBI GenBank and open reading frames within 40 ORFs on either side of NRPS B were analyzed. A total of 1069 translated sequences were subjected to an all vs. all blast and assembled into a sequence similarity network with an E value limit of 10⁻⁵ and alignment score of 50 using EFI-Enzyme Similarity Tool. The network was visualized using Cytoscape (version 3.7.1, from the National Resource of Network Biology). From the initial network five genomes were selected as having enough clustered proteins for a full BGC and were assembled into a more targeted SSN using an E value limit of 10⁻⁵ and alignment scores of 25 and 50. Manual analysis was complemented with antiSMASH 4.0 using the following: FMIB01000002.1 (M. chersina strain DSM 44151, cluster 1), NZ_CP016174 (A. orientalis strain B-37, cluster 13) NZ_ASJB01000042 (A. orientalis strain DSM 40040), NZ_KB913037 (A. balhimycina FH 1894 strain DSM 44591, clusters 1, 28), NZ_LT629775 (Streptomyces sp. TLI_053, cluster 18).

Bacterial strains and culture conditions. Micromonospora chersina DSM 44151 was purchased from the ATCC and cultivated as reported by Lam et al.65 Briefly, freeze-dried Micromonospora chersina DSM 44151 was reconstituted and grown on ISP 2 agar plates at 26° C. for 4 days until spore formation was visible. Spores were collected according to established protocols and used to inoculate 100 mL of seed medium 53 (10 g L⁻¹ fish meal; 30 g L⁻¹ dextrin; 10 g L⁻¹: lactose; 6 g L⁻¹ CaSO₄; and 5 g L⁻¹ CaCO₃) in a 250 mL culture flask, which was incubated for 7 days at 28° C. with orbital agitation at 250 rpm. Frozen vegetative stocks of M. chersina were prepared by mixing the seed culture suspension with an equal volume of 20% glycerol/10% sucrose, which was subsequently aliquoted, flash frozen with liquid nitrogen, and stored at −80° C.

Amycolatopsis orientalis DSM 40040 was purchased from the Leibniz Institute DSMZ. Freeze-dried A. orientalis was reconstituted in ISP I medium and plated onto ISP II agar plates. Plates were incubated at 26° C. for 5 days, after which the lawn of bacteria was lifted by adding sterile water (1 mL) and scraping gently with a sterile cell spreader. The suspension was used to inoculate 40 mL of vancomycin seed medium (5 g L⁻¹ glucose; 10 g L⁻¹ starch; 5 g L⁻¹ peptone; and 2 g L⁻¹ yeast extract) in a 250 mL culture flask, which was incubated for 2 days at 30° C. with orbital agitation at 220 rpm. Frozen vegetative stocks were prepared by mixing the seed culture suspension with an equal volume of 80% glycerol, which was subsequently aliquoted, flash frozen in liquid nitrogen, and stored at −80° C.

Amycolatopsis balhimycina FH 1894 DSM 44591 was purchased from the Leibniz Institute DSMZ. Freeze-dried A. balhimycina was reconstituted in GYM Streptomyces liquid medium and plated onto GYM Streptomyces agar plates. Agar plates were incubated at 28° C. for 4 days, after which the lawn of bacteria was lifted by adding sterile water (1 mL) and scraping gently with a sterile cell spreader. The suspension was used to inoculate 25 mL of tryptic soy broth in a 125 mL culture flask, which was incubated for 2 days at 28° C. with orbital agitation at 220 rpm. Frozen vegetative stocks were prepared by mixing culture suspension with an equal volume of 80% glycerol, which was subsequently aliquoted, flash frozen in liquid nitrogen, and stored at −80° C.

Antibiotic production screening in M. chersina DSM 44151. To prepare the seed culture, a frozen aliquot of M. chersina vegetative stock (4 mL) was thawed on ice, then used to inoculate a 500 mL baffle flask containing 100 mL of medium 53 and was incubated at 28° C. for 7 days with shaking at 250 rpm. For antibiotic production, seed culture (4 mL) was used to inoculate a 500 mL flask containing 100 mL of each of following media: dynemicin production media H881 (10 g L⁻¹ starch; 5 g L⁻¹ Pharmamedia; 1 g L⁻¹ CaCO₃; 0.05 g L⁻¹ CuSO₄; and 0.5 mg L⁻¹ NaI); H881 media with chicken oil (14 mL L⁻¹); H881 media with glucose (30 g L⁻¹); enduracidin growth media (80 g L⁻¹ corn flour; 30 g L⁻¹ corn gluten meal; 5 mL L⁻¹ corn steep liquor; 3 g L⁻¹ ammonium sulfate; 1 g L⁻¹ NaCl; 10 mg L⁻¹ ZnCl₂; 10 g L⁻¹ lactose; 10 mL L⁻¹ potassium lactate; and 14 mL L⁻¹ chicken oil), or ramoplanin production media (50 g L⁻¹ starch; 30 g L⁻¹ glucose; 30 g L⁻¹ soy flour; 10 g L⁻¹ CaCO₃; 5 g L⁻¹ leucine). The chicken oil supplement was prepared by defatting 1 whole roasting chicken (Harris Teeter, Inc.), rendering the isolated fat and skin at 350° C. for 15 min, cooling the mixture to rt, and clarifying the oil by centrifugation (15 min, 4,000 rpm, 4° C.). The oil was stored in the dark at 4° C. for up to 2 days prior to use.

Production cultures of M. chersina were grown at 28° C., 250 rpm for 12-21 days. Antibiotic production was monitored by MALDI-TOF MS screening. For screening, cell culture aliquots (6 mL) were pelleted by centrifugation at 5000 rpm for 15 minutes at 4° C. The supernatant was separated from the cell pellet by decantation and the supernatant fraction was extracted with ethyl acetate, and the organic fraction was separated, dried with sodium sulfate, and freed of solvent under vacuum. Both the aqueous and organic fractions were analyzed by MALDI-TOF MS analysis for production of secondary metabolites in the 2000-3000 Da MW range. Similarly, the production culture aliquot cell pellet was resuspended in acidic aqueous MeOH/H₂O (66:33 v/v; pH 3, 6 mL), stirred at rt for 3 h to affect cell lysis, centrifuged (5000 rpm, 10 min, 4° C.), and the supernatant was decanted and extracted with EtOAc as above. Both the aqueous and organic fractions were analyzed by MALDI-TOF MS. The antibiotic peptide was observed in the aqueous fraction of the extracted cell pellet, which was used for further analyses.

Antibiotic production screening in A. orientalis and A. balhimycina. A frozen vegetative stock of A. orientalis was used to inoculate an ISP II agar plate and incubated at 30° C., and a frozen vegetative stock of A. balhimycina was used to inoculate a GYM Streptomyces agar plate and incubated at 28° C. After 4 days, a single plate was used to inoculate a 50 mL seed culture by adding sterile water (1 mL) and lifting bacteria with a sterile cell spreader. The seed culture for A. orientalis was ISP medium I or vancomycin seed medium, and the seed culture for A. balhimycina was GYM Streptomyces medium or tryptic soy broth. Seed cultures were incubated at 28° C. with orbital agitation at 220 rpm for 2 days, then used to inoculate a 250 mL flask containing 50 mL of production media at 5% v/v. Production cultures were grown at 28° C. with orbital shaking at 220 rpm for 10 days, with aliquots removed for extraction on days 4, 7, and 10.

Culture media investigated for ramoplanin congener production from A. balhimycina included the following: GYM Streptomyces medium; ISP I liquid medium; ramoplanin production medium; and H881 medium. Culture media investigated for ramoplanin congener production from A. orientalis included the following: vancomycin production medium (20 g L⁻¹ glucose; 5 g L⁻¹ peptone; 0.75 g L⁻¹ MgSO₄; 1 g L⁻¹ NaCl; 0.5 g L⁻¹; and 1× trace metal solution) ramoplanin production medium; and H881 medium. Cell culture aliquots (6 mL) were screened as described for M. chersina. No positive hits were identified.

Large scale production, isolation, and purification of chersinamycin from M. chersina DSM 44151. For large scale production of chersinamycin from M. chersina, 20 mL of seed culture was used to inoculate 2 L baffled flasks containing 500 mL H881 media and grown at 28° C., 250 rpm for 12 days. Cells were pelleted by centrifugation, resuspended in acidic aqueous MeOH (300 mL), stirred at rt for 3 h at rt, then centrifuged to remove cellular debris as described above. The supernatant was extracted with EtOAc (3×300 mL) to remove organic-soluble metabolites. The aqueous layer was freeze-dried, dissolved in an H₂O/MeCN mixture, and subjected to RP-HPLC using a Jupiter C18, 250×21.2 mm column with a linear gradient of 20-50% B over 30 minutes, where solvent A is 0.1% TFA in H₂O and B is 0.06% TFA in MeCN. A second HPLC purification was performed using a Vydac C18 250×10 mm column with the same solvent system as above and a linear gradient of 20-35% B over 50 minutes to yield pure chersinamycin in 1 mg L⁻¹ quantities from the starting cell culture.

Macrolactone selective hydrolysis. Triethylamine (3 μL) was added to chersinamycin dissolved in water (0.115 μmol, 297 μL) to give 1% (v/v) TEA. The solution was allowed to sit at room temperature for one hour, and then analyzed by MALDI-TOF. After determining that the reaction had gone to completion by complete consumption of the starting material, the reaction mixture was dried and reconstituted in a water/acetonitrile mixture for further MS/MS analyses. Acyclic chersinamycin ESI-MS (m/z): [M+2H]²⁺ calcd for C₁₁₉H₁₆₀ClN₂₁O₄₂, 1296.044; found, 1296.044

Catalytic hydrogenation of the N-acyl lipid. The procedure for catalytic hydrogenation of the N-acyl lipid was modified from that described by Ciabatti and Cavalleri. Briefly, to a glass conical microvial charged with either ramoplanin A2 or chersinamycin (2 mg), MeOH/H₂O (10:90, v/v, 389 μL) was added and the solution was stirred at rt to facilitate dissolution. Once dissolved, Pd/C (2.5% w/w) was added (1 mg, 5.0 mol %), the flask was evacuated under vacuum, flushed with argon, and then the reaction mixture was placed under an atmosphere of H2 and stirred and monitored by analytical HPLC. After 8 h, additional Pd/C (2.5%, 1 mg) was added and the mixture stirred overnight under an H2 atmosphere. The reactions were diluted with MeOH/H₂O (10:90, v/v, 389 μL), filtered through Celite™, dried under vacuum, and analyzed by MALDI-TOF. A mass shift indicated a change from ramoplanin A2 (MALDI-TOF MH 2553.500) to tetrahydroramoplanin A2 (MALDI-TOF MH 2557.731). No mass shift was observed for chersinamycin (MALDI-TOF MH 2573.404).

Advanced Marfey's analysis of chersinamycin and ramoplanin. To facilitate the hydrolysis of chersinamycin and ramoplanin for advanced Marfey's analysis, to a thick walled glass vial (10 mL) containing either lyophilized chersinamycin (0.8 mg, 311 μmol) or ramoplanin (1 mg, 392 μmol) was added freshly prepared 6 M HCl (200 μL). After flushing the vial with Ar for 20 min, the vial was sealed and heated at 110° C. for 18 hrs. The reaction mixtures were cooled, evaporated under a stream of N2, dissolved in TEA/H₂O (25:75, v/v, 100 μL), transferred to a 5 mL round bottom flask, and evaporated under reduced pressure to dryness. The latter sequence was repeated 2 additional times. The resulting residue was dissolved in H₂O (75 μL), sodium bicarbonate (1M, 40 μL) and TEA (25 μL) were added, and the mixture was transferred to a 1.7 mL amber Eppendorf tube. Marfey's reagent (1.4 mg) in acetone (100 μL) was added and the mixture was heated for 1 h at 40° C. with periodic vortexing. After cooling to rt, HCl (2M, 10 μL) was added and the reaction mixture was dried overnight in a vacuum desiccator. For HPLC analysis, dried reaction mixtures were dissolved in DMSO (0.5 mL). A 50 μL aliquot was used to make a 1:1 dilution in water and filtered through a 0.2 μm syringe filter. RP-HPLC-MS analysis was performed with at Kintex 2.6 μm EVO-C18, 100×3 mm column with a gradient of 5-50% B over 40 minutes, where solvent A was 100:3:0.3 H₂O/MeOH/TFA and solvent B was 100:3:0.3 MeCN/H₂O/TFA. ESI-MS for FDAA-amino acids was performed in negative ion mode.

Structural determination by 1D and 2D NMR and ESI-MS/MS. Pure chersinamycin (3 mg, 2.6 mM) was dissolved in 4:1 H₂O/DMSO-d6 (v/v) or 4:1 D₂O/DMSO-d6 at pH 4.56. Homonuclear experiments were acquired with a spectral width of 11 ppm. Mixing times of 80 and 500 ms were used for TOCSY and NOESY spectra, respectively. Solvent suppression was employed at 2.50 ppm (DMSO) and 4.54 ppm (H₂O) and spectra were referenced to DMSO. For ESI-MS/MS analysis, pure cyclic and acyclic peptides dissolved in 4:1 H₂O/MeCN (v/v) were diluted 1:20 with 1:1 H₂O/MeCN (v/v) with 0.2% formic acid and infused into a Fusion Lumos Orbitrap mass spectrometer at 2.5 μL min⁻¹. Data was collected at 120 K for full MS scans and 30 K for MS/MS scans. The intact peptide was subjected to MS/MS higher-energy C-trap dissociation (HCD) fragmentation in both the [M+2H]²⁺ and [M+3H]³⁺ charge states.

Genetic and biochemical confirmation of antibiotic production by the predicted chersinamycin BGC. The M. chersina Dpg deletion mutant strain APKS7 was prepared as previously described and stored at −80° C. as frozen mycelial stocks. To assess the ability of M. chersina APKS7 to produce chersinamycin, a frozen aliquot (100 μL) of mycelia was thawed on ice, plated onto medium 53 agar and incubated at 28° C. for 5 days. Sterile liquid medium 53 was added to the plate (2 mL) and the plate was scraped to resuspend the cells. This suspension was added to a sterile culture flask (125 mL) containing medium 53 (50 mL), and the mixture was incubated for 7 days at 28° C. with shaking at 250 rpm. An aliquot of this seed culture (2 mL) was used to inoculate H881 media (50 mL) in a 250 mL sterile culture flask, which was incubated at 28° C. for 12 days with shaking (250 rpm). Following centrifugation, the production cell pellet was extracted with acidic aqueous MeOH/H₂O (66:33 v/v; pH 3, 50 mL) for 3 hours at rt. Cell debris was removed by centrifugation and the supernatant was subjected to HPLC-MS analysis for validation of the absence of detectible chersinamycin. To restore chersinamycin production through chemical complementation, M. chersina strain APKS7 was fermented in H881 production media that was supplemented with racemic (R,S)-3,5-Dpg (1 mM, Millipore-Sigma). Production cultures were incubated identically as above for 12 days at 28° C. with shaking at 250 rpm, the cell pellets were isolated by centrifugation, and then extracted and analyzed by HPLC-MS.

Minimal inhibitory assays. Antibacterial activity of chersinamycin and positive controls (vancomycin, ampicillin, and ramoplanin A2) were determined by the broth microdilution assay method. Briefly, bacterial strains were grown in cation-adjusted Mueller-Hinton broth. A microtiter plate was prepared by coating wells in 0.2% BSA, and antimicrobial peptides were added with 2-fold dilution steps ranging from 64-0.125 μg mL⁻¹. Bacteria was added to a final concentration of 10⁵ colony forming units and final volume of 100 μL. Plates were incubated at 37° C. for 24 hours, and the MIC was read as the lowest peptide concentration for which no bacterial growth was visualized. Reported values are the average of two replicates.

### Accession Codes

Ramoplanin biosynthetic gene cluster, Accession DD382878; Enduracidin biosynthetic gene cluster, DQ403252; Micromonospora chersina DSM 44151, Accession FMIB01000002.1; Amycolatopsis orientalis strain B-37, Accession NZ_CP016174; Amycolatopsis orientalis DSM 40040=KCTC 4912, Accession NZ_ASJB01000042; Amycolatopsis balhimycina FH 1894 DSM 44591, Accession NZ_KB913037; Streptomyces sp. TLI_053, Accession NZ_LT629775; Micromonospora sp. MH33, Accession NZ_MUYZ00000000.1; Amycolatopsis thailandensis strain JCM 16380, Accession NZ_NMQT00000000.1; Actinomadura madurae LIID-AJ290, Accession NZ_AW0002000001.1; Actinomadura madurae strain DSM 43067, Accession NZ_FOVH00000000.1; Streptomyces vietnamensis strain GIM4.0001, Accession NZ_CP010407.1; Streptomyces sp. GP55, Accession NZ_PJMT01000001.1; Streptomyces cinnamoneus strain ATCC 21532, Accession NZ_NHZ000000000.1; Streptomyces cinnamoneus strain DSM 41675, Accession NZ_PKFQ01000001.1

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

We claim:

1. A method for selecting a source organism of an antibiotic agent, the method comprising:
a. identifying a plurality of functionally significant structural motifs within at least one parent antibiotic agent;
b. selecting a plurality of probes, wherein each probe comprises a nucleotide sequence encoding an identified functionally significant structural motif or an amino acid sequence of an identified functionally significant structural motif;
c. identifying homologous proteins having at least 50% sequence identity to at least one probe or to the functionally significant structural motif encoded by at least one probe; and
d. selecting a source organism when the source organism comprises at least three homologous proteins.

2. The method of claim 1, wherein the at least one parent antibiotic agent is
a lipodepsipeptide antibiotic agent; and/or
a ramoplanin family antibiotic.

3. (canceled)

4. The method of claim 2, wherein the ramoplanin family antibiotic is ramoplanin or enduracidin.

5. (canceled)

6. The method of claim 1, wherein the functionally significant structural motifs are shared in two parent antibiotic agents, wherein the parent antibiotic agents are ramoplanin family antibiotic agents.

7. (canceled)

8. (canceled)

9. The method of claim 1, wherein the plurality of functionally significant structural motifs comprise a nonribosomal peptide synthetase (NRPS) or a domain thereof, a fatty acid adenylate forming ligase (FAAL) or a domain thereof, and/or an acyl carrier protein (ACP) or a domain thereof.

10. The method of claim 9, wherein the plurality of functionally significant structural motifs comprise at least two of NRPS A, NRPS B, NRPS C, NRPS D, the terminal thioesterase subdomain from NRPS C, FAAL, or ACP.

11. (canceled)

12. (canceled)

13. (canceled)

14. (canceled)

15. The method of claim 1, further comprising step e) determining whether the homologous proteins form a biosynthetic gene cluster; wherein determining whether the homologous proteins form a biosynthetic gene cluster comprises:
obtaining whole genome sequences for each selected source organism;
assembling a sequence similarity network comprising each whole genome sequence; and
determining whether a biosynthetic gene cluster is present within the sequence similarity network.

16. (canceled)

17. The method of claim 1, further comprising culturing at least one selected source organism to produce the antibiotic agent, and isolating the antibiotic agent from culture.

18. The method of claim 17, wherein the at least one selected source organism is determined to have a biosynthetic gene cluster that facilitates production of lipodepsipeptides.

19. The method of claim 17, wherein the antibiotic agent produced is a lipodepsipeptide antibiotic agent.

20. The method of claim 19, wherein the antibiotic agent produced is a ramoplanin congener.

21. The method of claim 20, wherein the antibiotic agent is chersinamycin.

22. (canceled)

23. (canceled)

24. The method of claim 17, further comprising purifying the isolated antibiotic agent.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A method of treating a bacterial infection in a subject comprising administering to the subject a ramoplanin congener obtained by the method of claim 20.

42. The method of claim 41, wherein the bacterial infection is an infection associated with one or more Gram-positive bacterium, wherein the infection is associated with Staphylococcus aureus, Staphylococcus epidermis, Staphylococcus saprophyticus, Staphylococcus haemolyticus, Staphylococcus hominis, Staphylococcus lugdunensis, Streptococcus pneumoniae, Streptococcus pyrogenes, Streptococcus agalactiae, Enterococcus faecium, Enterococcus faecalis, Bacillus anthracis, Bacillus cereus, Clostridium botulinum, Clostridium perfringens, Clostridium difficile, Clostridium tetani, Listeria monocytogenes, or Corynebacterium diptheria.

43. (canceled)

44. The method of claim 41, wherein the ramoplanin congener is chersinamycin.
